(19) United States
(12) Patent Application Publication
(10) Pub. No.: US 2026/0325222 A1
(43) Pub. Date: Oct. 22, 2026

(54) SYSTEM AND METHOD FOR AUGMENTED REALITY LAPAROSCOPIC PLANNING, NAVIGATION, AND VISUALIZATION

(71) Applicant: MEDIVIEW XR, INC., Cleveland, OH (US)

(72) Inventors: Jeff Yanof, Solon, OH (US); Seyedsina Shirinpour, Minneapolis, MN (US); Peter Nicholas Braido, Wyoming, MN (US); Ross Daniel Hinrichsen, Plymouth, MN (US); Cameron Nicholas Mar, Cleveland Heights, OH (US)

(21) Appl. No.: 19/634,682

(22) Filed: Mar. 31, 2026

Related U.S. Application Data
(60) Provisional application No. 63/780,545, filed on Mar. 31, 2025.

(51) Int. Cl. A61B 34/20; A61B 17/00; A61B 34/10; A61B 90/00

(52) U.S. Cl. CPC A61B 34/20; A61B 17/00234; A61B 2017/00203; A61B 2017/00725; A61B 2034/107; A61B 2034/2063; A61B 2034/2065; A61B 2090/365; A61B 2090/378

(57) ABSTRACT

An augmented reality system for laparoscopic procedures includes a tracking system that tracks positions and orientations of laparoscopic instruments and an ultrasound probe. A computer system receives pre-operative imaging data and generates three-dimensional representations of anatomical structures. The computer system receives tracking data and ultrasound imaging data, determines the imaging plane position and orientation of the ultrasound probe, and generates ultrasound flashlight projections displaying ultrasound imaging data positioned in three-dimensional space corresponding to the imaging plane. The computer system generates holographic light ray projections representing trajectories of the laparoscopic instruments. An augmented reality display system displays the ultrasound flashlight projections, holographic light ray projections, and three-dimensional representations of anatomical structures. The system enables pre-operative port placement planning using virtual lap arms defining trocar insertion paths and intraoperative surgical navigation with real-time visualization.

### CROSS-REFERENCE TO RELATED APPLICATIONS

This application claims the benefit of U.S. Provisional Application No. 63/780,545, filed on Mar. 31, 2025. The entire disclosure of the above application is incorporated herein by reference.

### FIELD

The present technology relates to surgical navigation and visualization systems, and, more particularly, to augmented reality systems for laparoscopic procedures utilizing electromagnetic tracking and holographic projections for improved surgical instrument guidance and ultrasound imaging visualization.

### INTRODUCTION

This section provides background information related to the present disclosure which is not necessarily prior art.

Laparoscopic surgery has become a widely adopted minimally invasive surgical approach that offers numerous benefits to patients. However, surgeons face challenges during these procedures, particularly related to visualization and instrument navigation. One of the primary difficulties is that surgeons must rely on two-dimensional monitor displays that can impair eye-hand coordination during procedures. The image orientation on these 2D monitors changes as devices are reoriented, and in some cases, the movement of devices on the monitor may appear inverted compared to the actual hand movements of the surgeon. While surgeons develop proficiency in positioning devices held by laparoscopic arms, the limitations of two-dimensional displays create inherent difficulties with depth perception and spatial awareness. The restricted field of view compounds these challenges, making precise instrument navigation more complex.

The integration of ultrasound imaging during laparoscopic procedures introduces additional complexity. Surgeons must cognitively align ultrasound data with their visualization of surgical instruments and anatomical structures. This mental mapping process adds to the cognitive load of performing intricate surgical maneuvers. The physical environment presents further complications, as patient positioning requirements often necessitate adjustments to table position and tilt. These positional changes affect the spatial relationships between surgical instruments and visualization systems, creating additional variables that surgeons must account for throughout the procedure. While electromagnetic tracking capabilities exist for monitoring surgical instruments and ultrasound probes, many 2D visualization systems do not fully leverage this tracking data. Additionally, laparoscopic cameras typically operate without position tracking, limiting opportunities for enhanced visualization techniques.

Accordingly, there is a continuing need for intra-operative planning, navigation and visualization systems that can address the challenges of two-dimensional display limitations, enhance spatial awareness, and provide integration of multiple imaging modalities while maintaining visual coordination with laparscopic arm movements as well as accurate spatial relationships between surgical instruments and anatomical structures.

### SUMMARY

In concordance with the instant disclosure, intra-operative planning, navigation and visualization systems that can address the challenges of two-dimensional display limitations, enhance spatial awareness, and provide integration of multiple imaging modalities while maintaining visual coordination with laparscopic arm movements as well as accurate spatial relationships between surgical instruments and anatomical structures have surprisingly been discovered.

The present technology includes articles of manufacture, systems, and processes that relate to augmented reality surgical navigation utilizing electromagnetic tracking, holographic projections of surgical instruments and ultrasound imaging, interactive visualization adjustments through voice commands and manual controls, and methods for enhancing eye-hand movement coordination during laparoscopic procedures through integrated display of tracked devices and imaging data.

In one embodiment, an augmented reality system for laparoscopic procedures comprises a tracking system that can track positions and orientations of a laparoscopic instrument and an ultrasound probe during a laparoscopic procedure. A computer system communicatively coupled to the tracking system can receive pre-operative imaging data from an imaging system and can generate a three-dimensional representation of an anatomical structure based on the pre-operative imaging data. The computer system can receive tracking data from the tracking system indicating the position and orientation of the laparoscopic instrument and the ultrasound probe, and can receive ultrasound imaging data from the ultrasound probe. The computer system can determine a position and orientation of an imaging plane of the ultrasound probe based on the tracking data and calibration data associated with the ultrasound probe, and can generate an ultrasound flashlight projection displaying the ultrasound imaging data positioned in three-dimensional space corresponding to the imaging plane. The computer system can further generate a holographic light ray projection representing a trajectory of the laparoscopic instrument based on the tracking data and calibration data associated with the laparoscopic instrument. An augmented reality display system communicatively coupled to the computer system can display the ultrasound flashlight projection, the holographic light ray projection, and the three-dimensional representation of the anatomical structure.

In another embodiment, a method of augmented reality-guided port placement planning for laparoscopic procedures can comprise receiving pre-operative imaging data from an imaging system at a computer system and processing the pre-operative imaging data to generate a three-dimensional representation of an anatomical structure and a skin surface hologram representing an external body surface of a patient. The method can include displaying, through an augmented reality display system, the three-dimensional representation of the anatomical structure and the skin surface hologram, and identifying an anatomical target location within the three-dimensional representation of the anatomical structure. The method can further comprise generating a plurality of virtual lap arms, each virtual lap arm defining a proposed laparoscopic instrument insertion path, each virtual lap arm including a proximal end positioned at the anatomical target and a distal end extending to intersect with the skin surface hologram. The method can include determining laparoscopic instrument trajectories for each of the virtual lap arms and generating a plurality of virtual port markers at intersection points where the virtual lap arms meet the skin surface hologram. The virtual lap arms, the laparoscopic instrument trajectories, and the virtual port markers can be displayed through the augmented reality display system, and a port placement plan including positions of the virtual lap arms, the virtual port markers, and the laparoscopic instrument trajectories can be stored.

In a further embodiment, a method of augmented reality-guided visualization for laparoscopic procedures can comprise tracking a position and an orientation of each one of a laparoscopic instrument and an ultrasound probe using a tracking system during a laparoscopic procedure. The method can include receiving tracking data from the tracking system at a computer system and receiving ultrasound imaging data from an ultrasound probe at the computer system. The method can further comprise determining an imaging plane position and orientation of the ultrasound probe based on the tracking data and calibration data associated with the ultrasound probe, and generating an ultrasound flashlight projection displaying the ultrasound imaging data positioned in three-dimensional space corresponding to the imaging plane. The method can include determining instrument axes for the laparoscopic instrument based on the tracking data and calibration data associated with the laparoscopic instrument, and generating a holographic light ray projection extending along the instrument axes. The ultrasound flashlight projection and the holographic light ray projections can be displayed through an augmented reality display system, and the ultrasound flashlight projection and the holographic light ray projection can be continuously updated as the laparoscopic instruments and the ultrasound probe move during the procedure.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a block diagram of a system for augmented reality-guided laparoscopic procedures including an augmented reality display system, a tracking system, laparoscopic instruments, an ultrasound probe, a computer system, and imaging systems.

FIG. 2 is a perspective view of multiplanar reformatting slice projections, an anatomical target marker, target intersection markers, and three-dimensional representations of anatomical structures displayed through the augmented reality display system.

FIG. 3 is a perspective view similar to FIG. 2, further depicting a skin surface hologram, virtual lap arms, laparoscopic instrument trajectories, and virtual port markers displayed for port placement planning.

FIG. 4 is a perspective view similar to FIG. 3, further depicting a bounding box control interface positioned at a proximal end of a virtual lap arm to enable interactive adjustment of laparoscopic instrument trajectories.

FIG. 5 is a perspective view similar to FIG. 4, depicting collision detection between laparoscopic instrument trajectories and critical anatomical structures to be avoided during port placement.

FIG. 6 is a perspective view depicting an ultrasound flashlight projection displayed on a patient's body in spatial alignment with an ultrasound probe.

FIG. 7 is a perspective view of a laparoscopic instrument with a mounting bracket attached thereto.

FIG. 7A is an enlarged detailed view of the mounting bracket of FIG. 7.

FIG. 8 is a diagram depicting bounding box control directions including pitch, yaw, and roll adjustments for manipulation of holographic projections in float projection mode.

FIGS. 9A-9C are flowcharts depicting a method of providing augmented reality guidance for laparoscopic procedures.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the terms “interventional device” or “tracked instrument” refers to a medical instrument used during a medical procedure.

As used herein, the term ‘tracking system’ refers to something used to observe one or more objects undergoing motion and supply a timely ordered sequence of tracking data (e.g., location data, orientation data, or the like) in a tracking coordinate system for further processing. As an example, the tracking system can include electromagnetic tracking that can observe an interventional device equipped with a tracker, optical tracking that can detect reflective markers or light sources, fiber optic shape sensing for flexible catheters and instruments within the body, electro-anatomical mapping, impedance-based tracking, or other tracking modalities that can observe the interventional device as it enters, moves through and/or in, and exits a patient's body, as well as while outside of the patient's body.

As used herein, the term “tracking data” refers to information recorded by the tracking system related to an observation of one or more objects undergoing motion.

As used herein, the term “head-mounted device” or “headset” or “HMD” refers to a display device, configured to be worn on the head, that has one or more display optics (including lenses) in front of one or more eyes. These terms may be referred to even more generally by the term “augmented reality system” or “mixed reality system,” where mixed reality refers to interactive environments that combine physical objects with their related data in a manner similar to Medical Internet of Things (MIOT) applications. It should be appreciated that the term “augmented reality system” is not limited to display devices configured to be worn on the head. In some instances, the head-mounted device can also include a non-transitory memory and a processing unit. Examples of suitable head-mounted devices include Microsoft HoloLens® head-mounted device (Microsoft, Redmond, Washington), Magic Leap 2 (Magic Leap, Inc.), and DigiLens (DigiLens Inc.) devices.

As used herein, the terms “imaging system,” “image acquisition apparatus,” “image acquisition system” or the like refer to technology that creates a visual representation of the interior of a patient's body. For example, the imaging system can be a computed tomography (CT) system, a fluoroscopy system, positron emission computed tomography, magnetic resonance imaging (MRI) system, an ultrasound (US) system including contrast agents, color flow doppler, 4D ultrasound with volume rendering capabilities, or the like.

As used herein, the terms “coordinate system” or “augmented reality system coordinate system” or “augmented reality system coordinates” refer to a 3D Cartesian coordinate system that uses one or more numbers to determine the position of points or other geometric elements unique to the particular augmented reality system or image acquisition system to which it pertains. This includes coordinate data from both internal sensors within the headset (such as inertial measurement units and optical sensors) as well as external sensors that may be attached to the headset and/or user for enhanced tracking capabilities. For example, 3D points in the headset coordinate system can be translated, rotated, scaled, or the like, from a standard 3D Cartesian coordinate system.

As used herein, the terms “image data” or “imaging dataset” or “imaging data” refers to information recorded in 3D by the imaging system related to an observation of the interior of the patient's body. For example, the “image data” or “imaging dataset” can include processed two-dimensional or three-dimensional images or models such as imaging images, e.g., represented by data formatted according to the Digital Imaging and Communications in Medicine (DICOM) standard or other relevant imaging standards.

As used herein, the terms “imaging coordinate system” or “image acquisition system coordinate system” refers to a coordinate system that uses one or more numbers to determine the position of points or other geometric elements unique to the particular imaging system. This includes three-dimensional orthogonal coordinate systems such as Cartesian coordinates, cylindrical coordinates, spherical coordinates, bipolar cylindrical coordinates, bispherical coordinates, confocal ellipsoidal coordinates, and other orthogonal curvilinear coordinate systems, as well as quaternions for representing three-dimensional rotations. For example, points and vectors in the imaging coordinate system can be translated, rotated, scaled, or the like, to the Augmented Reality system (head mounted displays) coordinate system.

As used herein, the terms “hologram”, “holographic,” “holographic projection”, or “holographic representation” refer to a computer-generated image stereoscopically projected through the lenses of a headset. Generally, a hologram can be generated synthetically (in an augmented reality (AR)) and is not a physical entity.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two or more things being linked or coordinated. For example, a coil-sensor can be integrated with an interventional device.

As used herein, the term “real-time” or “near-real time” or “live” refers to the actual time during which a process or event occurs. In other words, a real-time event is done live (within milliseconds so that results are available immediately as feedback). For example, a real-time event can be represented within 100 milliseconds of the event occurring.

As used herein, an exemplary ultrasound probe is a handheld transducer device that emits and receives high-frequency sound waves to generate diagnostic images of internal body structures. It converts electrical signals to acoustic waves and vice versa via piezoelectric elements. Such probes are referenced across imaging, biopsy guidance, and therapeutic applications. The Philips X5-1 matrix array probe and the GE Voluson E8 transducer probe are examples of ultrasound probes that may be employed in the present disclosure.

As used herein, the term spatial “registration” refers to steps of transforming tracking and imaging dataset associated with virtual representation of tracked devices-including holographic guides, applicators, and ultrasound image stream- and additional body image data for mutual alignment and correspondence of said virtual devices and image data in the head mounted displays coordinate system enabling a stereoscopic holographic projection display of images and information relative to a body of a physical patient during a procedure, for example, as further described in U.S. patent application Ser. No. 10/895,906 to West et al., and also applicant's co-owned U.S. patent application Ser. No. 18/674,102 to Yanof et al., U.S. patent application Ser. No. 17/110,991 to Black et al. and U.S. Pat. No. 11,701,183 to Martin III et al., the entire disclosures of which are incorporated herein by reference.

The present technology improves surgical navigation and visualization during laparoscopic procedures by addressing challenges with eye-hand coordination through holographic projections aligned with physical instruments, imaging, and the patient's body, thereby enhancing spatial awareness through optical and/or electromagnetic tracking of surgical instruments and ultrasound probes, providing flexible visualization options including “float projection mode” and “in situ” projections that maintain geometric relationships between virtual representation of the instruments and imaging while improving viewing angles, allowing for ergonomic standing position, and enabling intuitive adjustment of holographic elements through voice commands, manual, and automated controls, and facilitating the integration of multiple imaging modalities to reduce cognitive load during surgical procedures. The augmented reality system 102 can implement comprehensive tracking and analytics capabilities to optimize surgical procedures. The system can track and record virtual video feed positions, device locations, and port placements from initial setup through completion, enabling both real-time analysis and post-procedure review. This tracking data allows the system to develop user preferences and predict optimal outcomes based on patient-specific factors including anthropometry and morphology. Through analysis of accumulated procedural data, the system can prescribe optimal configurations for port locations, device positioning, video stream orientation, and DICOM image alignment over time. These analytics capabilities enable continuous refinement of surgical approaches while accounting for individual patient characteristics and surgeon preferences.

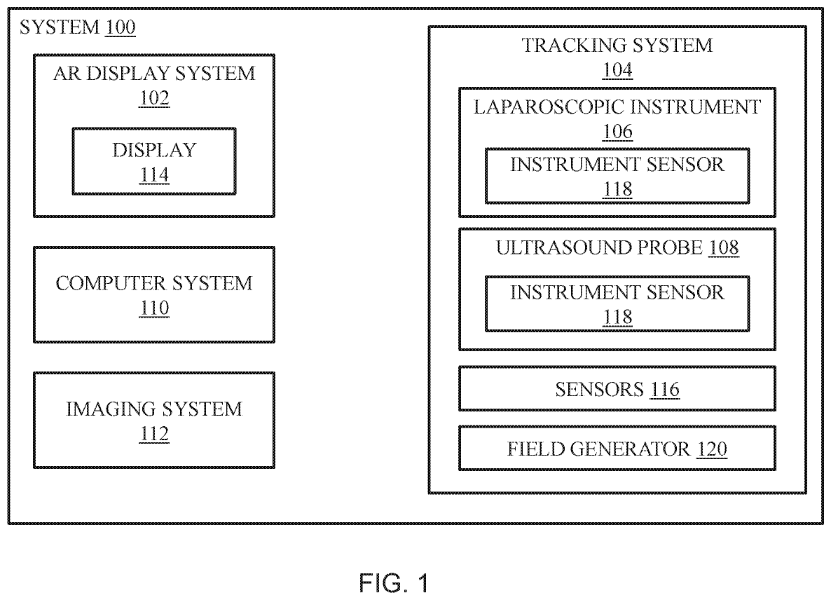

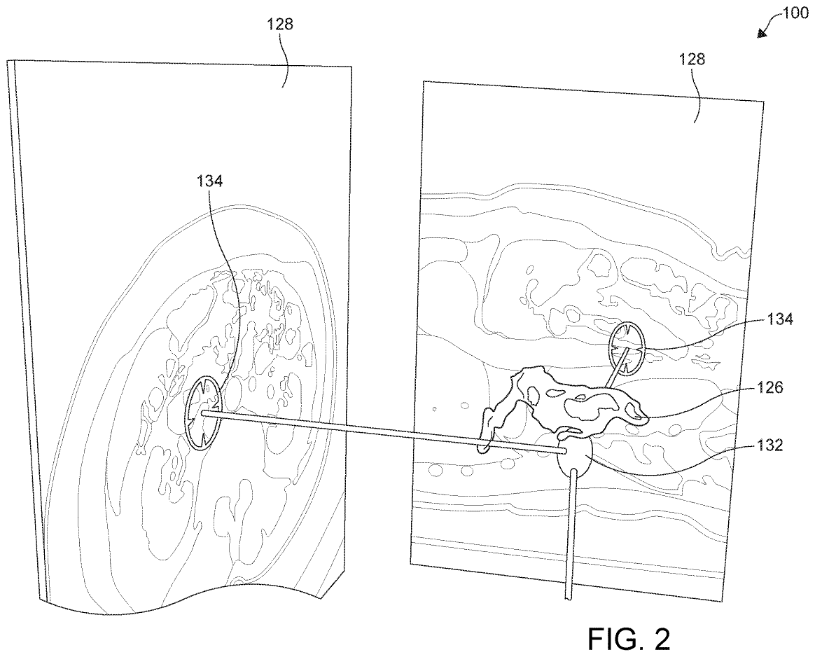

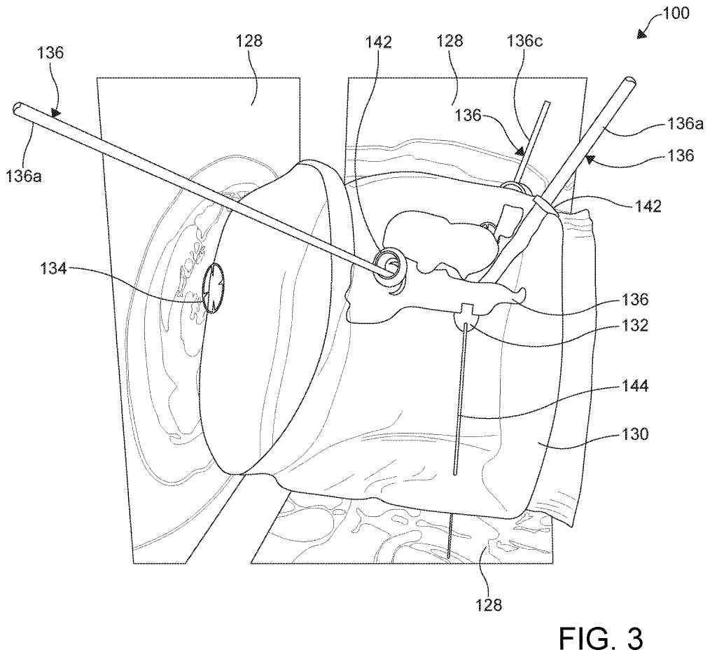

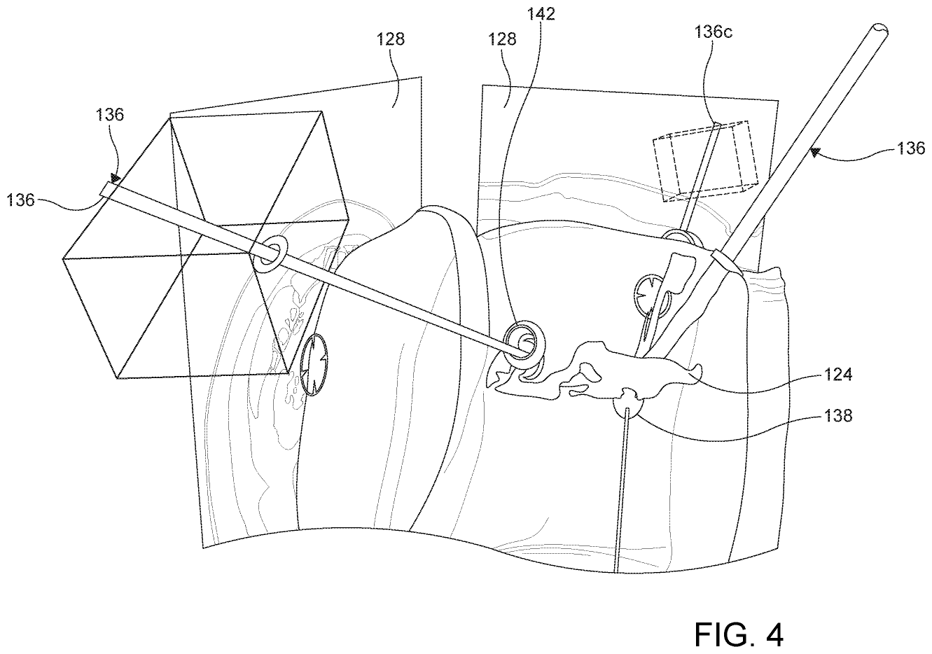

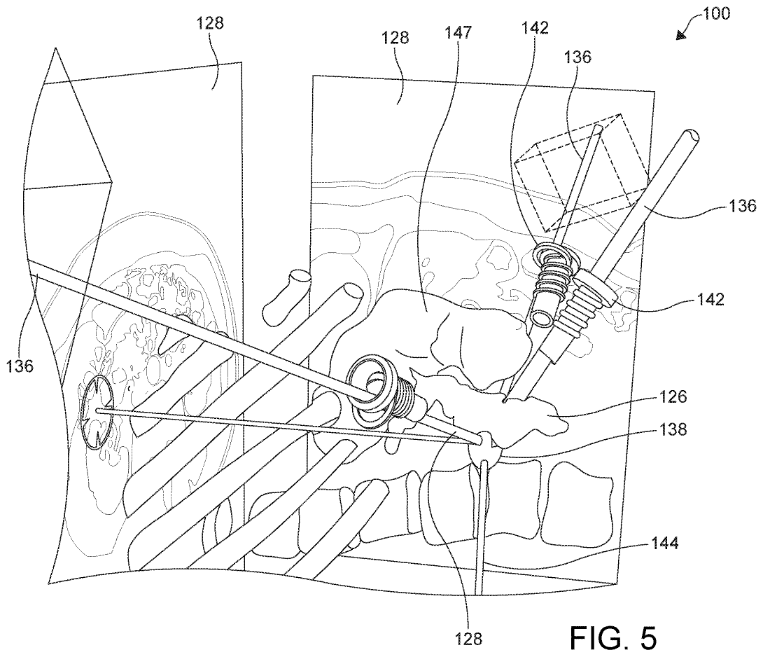

With reference to FIGS. 1-8, a system for laparoscopic navigation and visualization 100 is shown. The system 100 can include an augmented reality display system 102 and an optical and/or electromagnetic tracking system 104 that work together to provide real-time holographic guidance during laparoscopic procedures. The augmented reality system 102 can display holographic representations of tracked instruments, ultrasound imaging data, and pre-operative anatomical imaging overlaid on the surgical field, as non-limiting examples. The electromagnetic tracking system 104 can track the position and orientation of laparoscopic instruments 106 and an ultrasound probe 108, providing spatial data that enables the augmented reality system 102 to maintain accurate alignment between holographic projections and physical devices. The holographic projections can enable enhanced spatial visualization through stereoscopic projection, improving eye-hand coordination and spatial awareness during surgical navigation.

The augmented reality system 102 can be configured to display holographic projections including two-dimensional (2D) or three-dimensional (3D) representations of information for laparoscopic procedures. The holographic projections can include tracked device representations showing position and orientation of the one or more laparoscopic instruments 106 and ultrasound probe 108 being tracked by the tracking system 104. The holographic projections can include Holographic Light Ray (HLR) projections aligned with surgical instruments, representing instrument trajectories for laparoscopic instrument devices including trocars and ablation devices. The holographic projections can include ultrasound flashlight projections that display ultrasound imaging data extending from the tracked ultrasound probe 108 and overlaid on anatomy of a patient. The holographic projections can include three-dimensional depictions of anatomical features of the patient including organs, portions of organs, tissues, joints, bones, tumors, vasculature, and implants. The holographic projections can include laparoscopic port placement planning visualizations. The holographic projections can include visualization of a laparoscopic camera frustum showing the camera's field of view relative to tracked instruments and anatomical structures. The holographic projections can be displayed over a portion of the patient corresponding to the anatomical feature being visualized.

The augmented reality system 102 can be configured to operate in extended reality modalities including augmented reality (AR) where holographic content is overlaid on the physical world, virtual reality (VR) where camera passthrough enables viewing of the physical environment within an immersive virtual space, and mixed reality (MR) where physical and virtual elements are combined in an interactive environment. The augmented reality system 102 can transition between modalities based on procedural requirements.

The augmented reality system 102 can include a display 114 configured to present the holographic projections to an operator. In certain embodiments, the display 114 can include a head-mounted display device configured to present stereoscopic holographic content to an operator while maintaining visibility of the physical environment through optical see-through visualization. Optical see-through head-mounted displays can utilize transparent waveguide optics or holographic optical elements that enable virtual images to be overlaid on the physical world while allowing ambient light to pass through. The stereoscopic display can present different images to each eye, creating binocular disparity for depth perception of holographic elements in three-dimensional space. Head-mounted display embodiments can be implemented using devices such as Microsoft HoloLens, Microsoft HoloLens 2, or Magic Leap devices.

In alternative embodiments, the display 114 can include a non-head-mounted display device such as a computer monitor, tablet computer, smartphone, or other electronic display configured to present augmented reality visualizations through video see-through methods or as separate display windows. Video see-through implementations can capture the physical environment through cameras and overlay virtual content on the video feed. Optical see-through implementations can project virtual content onto transparent optical elements positioned in the operator's line of sight. The augmented reality system 102 can support multiple display types simultaneously, including head-mounted displays, handheld devices, and fixed position displays.

The augmented reality system 102 can include camera systems configured to capture images of the physical environment for optical tracking, spatial mapping, and hand gesture recognition. The camera systems can include visible light cameras, infrared cameras, and depth sensors. In head-mounted display embodiments, the camera systems can be positioned on the head-mounted display to provide overlapping fields of view covering the procedural workspace. In non-head-mounted display embodiments, the camera systems can be integrated into tablet computers, smartphones, or positioned as external cameras that capture the surgical field.

The augmented reality system 102 can be configured to receive and display pre-operative imaging data including Computed Tomography (CT), Magnetic Resonance Imaging (MRI), and Positron Emission Tomography (PET) scans. The augmented reality system 102 can display the imaging data through the holographic projections, enabling visualization of internal anatomical structures, tumors, vasculature, and other features in three-dimensional space. The augmented reality system 102 can display multiple imaging modalities simultaneously, enabling cross-referencing between different imaging datasets. The augmented reality system 102 can provide multiplanar reformatting (MPR) of imaging data, enabling visualization in arbitrary planes.

The augmented reality system 102 can include processing capabilities distributed across a central processing unit (CPU), a graphics processing unit (GPU), and a holographic processing unit (HPU) configured for spatial mapping and holographic display generation. The augmented reality system 102 can include memory components including random access memory (RAM), read-only memory (ROM), hard disk drives (HDD) or solid-state drives (SSD), and non-transitory computer-readable media storing instructions for system operation. In certain embodiments, processing capabilities can be distributed between the display 114 and external workstations, with tracking data processing performed on workstation computers and holographic rendering performed on processors of the display 114.

The augmented reality system 102 can be configured to enable interactive adjustment of the holographic projections through voice commands and manual controls. The holographic projections can be adjusted relative to the ultrasound probe 108, laparoscopic instruments 106, and operator orientation while maintaining spatial relationships between the holographic projections and physical devices through continuous tracking by the tracking system 104. Laparoscopic port placement planning visualizations generated pre-operatively can be stored and retrieved for display during procedures.

The tracking system 104 can include one or more sensors 116 configured to measure position and orientation of surgical instruments and medical devices, for example, laparoscopic instruments 106, and ultrasound probes 108 and a system processor that calculates tracking data from sensor signals. The sensors 116 can be configured to attach to or integrate with laparoscopic instruments 106, ultrasound probes 108, and other surgical tools to enable continuous monitoring of instrument positions and orientations throughout surgical procedures. The tracking system 104 can provide real-time tracking data in a timely ordered sequence, enabling the augmented reality system 102 to display holographic representations that maintain accurate spatial alignment with physical devices. The sensors 116 can be implemented as 5-degree-of-freedom (5DOF) sensors that measure position through three translational coordinates and partial orientation through two rotational coordinates, or as 6-degree-of-freedom (6DOF) sensors that measure position through three translational coordinates and full orientation through three rotational coordinates including pitch, yaw, and roll.

In one embodiment, the tracking system 104 can be implemented as an electromagnetic tracking system that includes an electromagnetic field generator that produces an electromagnetic field within a defined tracking volume, and one or more electromagnetic sensors 116 configured to measure position and orientation within the electromagnetic field. The system processor can calculate tracking data from the electromagnetic signals received by the electromagnetic sensors 116. The electromagnetic field generator can be implemented as a tabletop generator mounted beneath the patient table that moves with table adjustments, a planar generator positioned adjacent to the patient that remains stationary during table movements, or a portable field generator that can be repositioned as needed during procedures. The electromagnetic sensors 116 can be implemented as 5-degree-of-freedom (5DOF) electromagnetic coil sensors or as 6-degree-of-freedom (6DOF) electromagnetic coil sensors. Compatible electromagnetic tracking systems can include the Aurora system from Northern Digital Inc., Ascension trakSTAR, and similar electromagnetic navigation systems, as non-limiting examples.

In alternative embodiments, the tracking system 104 can utilize other tracking modalities including optical tracking that can detect reflective markers or light sources attached to surgical instruments, fiber optic shape sensing for flexible catheters and instruments within the body, electro-anatomical mapping systems that measure electrical impedance to determine catheter positions, impedance-based tracking systems, inertial measurement units (IMUs) that measure acceleration and angular velocity, acoustic tracking systems, or other tracking modalities. The tracking system 104 can also implement hybrid approaches combining multiple tracking modalities working together through sensor fusion algorithms to maintain registration accuracy. For example, the tracking system 104 can combine electromagnetic tracking of instruments with optical tracking through the built-in headset cameras detecting image markers and model targets positioned on tracking system components or anatomical reference points. The tracking system 104 can utilize optical tracking through headset-mounted cameras tracking optical trackers positioned on surgical instruments, or can combine inertial measurement unit data with electromagnetic tracking data to improve tracking robustness in challenging electromagnetic environments.

The system 100 can include a computer system 110 configured to receive data from the tracking system 104, the augmented reality system 102, one or more imaging systems 112, and the ultrasound probe 108, and process the data to generate the holographic projections. The computer system 110 can include one or more processors, memory, and communication interfaces configured to coordinate data flow between system components. In certain embodiments, the computer system 110 can be integrated within the augmented reality system 102. In alternative embodiments, the computer system 110 can be implemented as an external workstation in communication with the augmented reality system 102 and the tracking system 104.

The tracking system 104 can maintain continuous registration with the augmented reality system 102 through coordinate system transformations that relate tracked positions measured in the tracking system coordinate system to positions in the head-mounted display coordinate system. This registration enables co-projection and alignment of holographic elements with corresponding physical devices throughout surgical procedures. The system can maintain accurate spatial relationships between holographic projections and tracked instruments while accommodating user movement, viewing angle changes, and procedural dynamics. The coordinate system transformations can be computed through registration algorithms that align tracking coordinate systems with imaging coordinate systems and head-mounted display coordinate systems, enabling accurate holographic visualization of tracked instruments relative to pre-operative imaging data and physical patient anatomy.

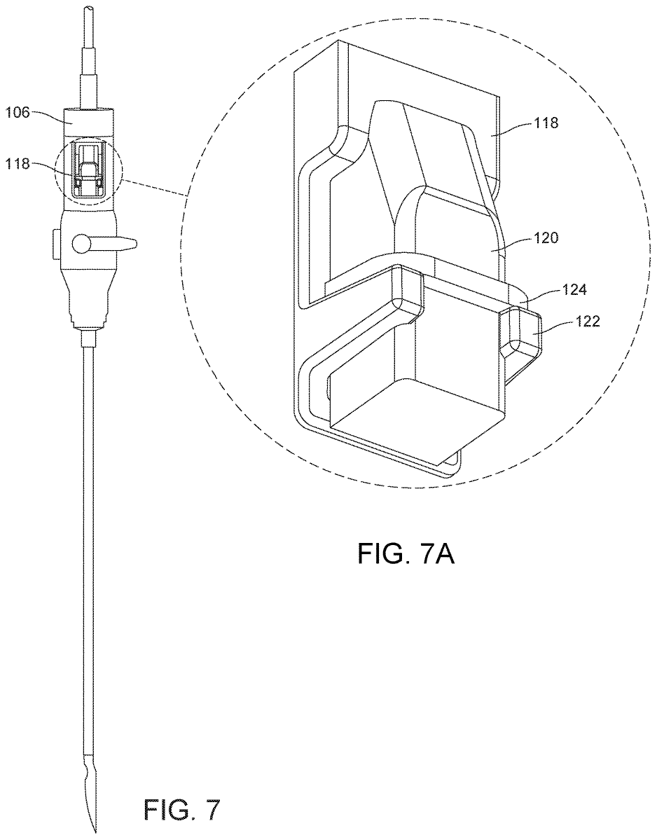

With reference to FIGS. 7 and 7A, the system 100 can be configured to integrate one or more sensors 116 of the tracking system 104 with the laparoscopic instruments 106 and the ultrasound probe 108 through mounting and sensor attachment infrastructure. The sensors 116 can include instrument sensors 122 configured to attach to surgical instruments. The system 100 can incorporate mounting brackets 118 configured to attach the instrument sensors 122 to the laparoscopic instruments 106, ultrasound probe 108, and other surgical devices used during procedures. The mounting brackets 118 can be engineered to securely attach to existing instruments while maintaining proper orientation and positioning for accurate tracking. This adaptability can enable healthcare facilities to leverage existing equipment investments while adding augmented reality guidance and navigation capabilities through the augmented reality system 102 without requiring wholesale equipment replacement.

The mounting brackets 118 can accommodate multiple device form factors and configurations. For ultrasound applications, the mounting brackets 118 can accommodate curvilinear probes, linear probes, phased array probes, and other ultrasound transducer configurations for the ultrasound probe 108. For laparoscopic applications, the mounting brackets 118 can accommodate rigid instruments, flexible instruments, and camera-based devices for the laparoscopic instruments 106 including endoscopic ultrasound devices, borescopes, videoscopes, and fiberscopes. The mounting brackets 118 can be configured to attach to various manufacturer-specific instrument models, enabling compatibility with currently deployed medical equipment across different healthcare facilities. Similarly, the mounting bracket 118 can be configured to attach instrument sensors 120 to various laparoscopic instruments 106 including trocars, graspers, ablation devices, and camera-based laparoscopic instruments. The mounting brackets 118 can be configured as sterilizable and reusable components that can be processed through standard surgical instrument sterilization protocols.

The one or more sensors 116 of the tracking system 104 can include one or more instrument sensors 120 configured to be attached to the laparoscopic instruments 106 and ultrasound probe 108. The instrument sensors 120 can be configured to be disposed within or on the mounting brackets 118, enabling tracking of the laparoscopic instruments 106 and ultrasound probe 108 during surgical procedures. The mounting brackets 118 can incorporate one or more attachment mechanisms 122 configured to secure the instrument sensors 120 to the mounting brackets 118 while enabling sensor removal and replacement. The attachment mechanisms 122 can include mechanical features such as snap-fit mechanisms, clamp structures, threaded connections, magnetic attachments, or adhesive interfaces. The attachment mechanisms 122 can be configured to ensure consistent and repeatable sensor placement, maintaining accurate spatial relationships between the instrument sensors 120 and the laparoscopic instruments 106 or ultrasound probe 108 to which they are attached. The attachment mechanisms 122 can enable selective removability of the instrument sensors 120, allowing the instrument sensors 120 to be detached for separate sterilization, battery replacement, or sensor reuse across multiple mounting brackets 118.

The instrument sensors 120 can include one or more structural features 124 configured to interface with corresponding features on the mounting brackets 118 to ensure consistent instrument sensor 120 positioning and orientation. The structural features 124 can include alignment features such as keyed grooves, alignment pins, shaped protrusions, or indexed surfaces that mate with corresponding receptacles, channels, or surfaces on the mounting bracket 118. The structural features 124 of the instrument sensors 120 can include positioning features such as backstops, shoulders, flanges, or stops that engage with corresponding surfaces on the mounting bracket 118 to define sensor insertion depth. The system 100 can utilize calibration data that defines geometric relationships between the instrument sensors 122 and reference points on the laparoscopic instruments 106 and ultrasound probe 108. Each mounting bracket 118 and instrument sensor 122 combination can have associated calibration data including coordinate offsets, rotational offsets, and transformation matrices that relate sensor coordinate systems to instrument coordinate systems. The calibration data can be determined during manufacturing, acquired during pre-operative planning procedures, performed prior to each surgical procedure, or updated periodically to maintain tracking accuracy. The calibration data can be stored in memory accessible to the tracking system 104. The calibration data can account for the physical dimensions of the mounting brackets 118, the position of the instrument sensors 122 within the mounting brackets 118, and the geometry of the laparoscopic instruments 106 and ultrasound probe 108 to which the mounting brackets 118 are attached.

The calibration data can be encoded in machine-readable formats and associated with specific device identifiers. In certain embodiments, the calibration data can be embedded in or attached to the mounting brackets 118 or instrument sensors 120 through machine-readable codes such as quick response (QR) codes, barcodes, radio-frequency identification (RFID) tags, or near-field communication (NFC) chips. When a device is prepared for use, the system 100 can read the embedded calibration data through optical scanning of QR codes or barcodes using the camera systems of the augmented reality system 102, RFID reading, or NFC communication and load the geometric offset values specific to that device and mounting bracket 118 combination. The calibration data can be updated over time as the laparoscopic instruments 106 and ultrasound probe 108 are used.

In alternative embodiments, the instrument sensors 120 can be directly integrated into the laparoscopic instruments 106 or ultrasound probe 108 during manufacturing rather than attached through separate mounting brackets 118. Integrated instrument sensors 120 can be embedded within instrument housings, removing the need for external mounting brackets 118. The integrated instrument sensors 120 can be calibrated during manufacturing to determine geometric relationships between the integrated instrument sensors 120 and reference points on the laparoscopic instruments 106 or ultrasound probe 108. The system 100 can utilize both bracket-mounted instrument sensors 120 and integrated instrument sensors 120, with calibration data associated with each configuration.

The instrument sensors 120 can be configured for wired or wireless communication with the tracking system 104. Wireless instrument sensors 120 can include battery power sources and wireless communication circuitry configured to transmit tracking data to the tracking system 104 through wireless protocols including Bluetooth, WiFi, or proprietary radio frequency communication. Wired instrument sensors 120 can be connected to the tracking system 104 processor or, in electromagnetic tracking embodiments, to the electromagnetic field generator through cables. The system 100 can include both wireless instrument sensors 120 and wired instrument sensors 120 within the same procedure, where some laparoscopic instruments 106 include wireless instrument sensors 120 while the ultrasound probe 108 or other laparoscopic instruments 106 include wired instrument sensors 120, and vice versa.

The instrument sensors 120 can be implemented using various sensor modalities. In electromagnetic tracking embodiments, the instrument sensors 120 can be electromagnetic coil sensors. In optical tracking embodiments, the instrument sensors 120 can include optical markers including reflective spheres or active light-emitting diodes (LEDs). In inertial tracking embodiments, the instrument sensors 120 can include inertial measurement units (IMUs) that measure acceleration and angular velocity. In acoustic tracking embodiments, the instrument sensors 120 can include acoustic sensors that measure ultrasonic signals. In infrared tracking embodiments, the instrument sensors 120 can include active or passive infrared emitters or receivers. The mounting brackets 118 can be configured to receive multiple instrument sensor 120 types on a single laparoscopic instrument 106 or the ultrasound probe 108, with the tracking system 104 configured to process data from multiple sensor modalities through sensor fusion algorithms.

The augmented reality system 102 and tracking system 104 can communicate through network configurations including local area networks (LAN), wide area networks (WAN), and wireless local area networks (WLAN). The tracking system 104 can transmit tracking data to the computer system through tracking system interfaces configured to receive position and orientation data for the instrument sensors 120. The tracking data can include position coordinates and orientation values for each of the instrument sensors 120 attached to the laparoscopic instruments 106 and ultrasound probe 108. The computer system 110 can process the tracking data by applying calibration data associated with each mounting bracket 116 and instrument sensor 120 combination to determine the position and orientation of the laparoscopic instruments 106 and ultrasound probe 108 in three-dimensional space.

The system 100 can communicate with imaging systems 112 through Digital Imaging and Communications in Medicine (DICOM) networking protocols to receive imaging datasets from imaging modalities or picture archiving and communication systems (PACS). The imaging datasets can include Computed Tomography (CT) scans, Magnetic Resonance Imaging (MRI) scans, Positron Emission Tomography (PET) scans, and other medical imaging data. The computer system 110 can be configured to receive the imaging datasets, process the imaging data to extract anatomical structures, and generate three-dimensional representations of the anatomical structures for display through the holographic projections. The system 100 can store imaging datasets in memory for retrieval and display during procedures.

The computer system 110 can be configured to continuously receive and process data from the tracking system 104, imaging systems 112, and the ultrasound probe 108, and update the holographic projections based on the received data. As the laparoscopic instruments 106 and ultrasound probe 108 move during procedures, the tracking system 104 can transmit updated tracking data to the computer system 110, and the computer system 110 can update the position and orientation of holographic representation of the laparoscopic instruments 106 and ultrasound probe 108 in the holographic projections. The augmented reality system 102 can render the updated holographic projections on the display 114 of the augmented reality system 102.

The system 100 can include data storage configured to store procedural data including pre-operative imaging datasets, calibration data for the mounting brackets 116 and instrument sensors 120, port placement plans generated pre-operatively, and recorded tracking data from procedures. The computer system 110 can retrieve stored data for display during procedures. Port placement plans generated pre-operatively can be retrieved and displayed during procedures to guide trocar insertion. The system 100 can store tracking data recorded during procedures for post-procedural review and analysis.

The computer system 110 can be configured to perform coordinate transformations to align holographic content with physical devices and anatomical structures. The tracking system 104 can provide position and orientation data for the instrument sensors 120 in a tracking coordinate system. The augmented reality system 102 can maintain a spatial coordinate system defining positions in three-dimensional space relative to the augmented reality system 102. The computer system 110 can apply transformation matrices to convert tracking data from the tracking coordinate system to the spatial coordinate system of the augmented reality system 102, enabling holographic representations of tracked devices to be displayed in alignment with the physical laparoscopic instruments 106 and ultrasound probe 108.

The computer system 110 can utilize the calibration data associated with each mounting bracket 120 and instrument sensor 120 combination to determine the position and orientation of instrument reference points relative to the instrument sensors 120. The calibration data can define geometric offsets between the instrument sensors 120 and functional portions of the laparoscopic instruments 106 and ultrasound probe 108, such as instrument tips, imaging planes, or tool axes. The computer system 110 can apply the calibration data during coordinate transformations to determine the position and orientation of the functional portions of the laparoscopic instruments 106 and ultrasound probe 108 for display through holographic content.

The augmented reality system 102 can perform spatial mapping to detect surfaces and objects in the physical environment and establish spatial anchors that maintain consistent positioning of holographic content relative to physical objects as the operator moves. The computer system 110 can register pre-operative imaging data to the patient anatomy through registration processes including anatomical landmark identification, surface registration, or fiducial marker registration, enabling holographic projections of anatomical structures to be aligned with the patient.

The augmented reality system 102 can be configured to provide laparoscopic surgical guidance using pre-operative tomographic imaging integrated with real-time tracking and holographic visualization. The computer system 110 can receive pre-operative imaging data from the imaging systems 112. The pre-operative imaging data can include Computed Tomography (CT) scans, Magnetic Resonance Imaging (MRI) scans, Positron Emission Tomography (PET) scans, or other tomographic imaging data. The computer system 110 can process the pre-operative imaging data to generate the three-dimensional representations of anatomical structures 126 including organs, vessels, bones, tumors, and other structures relevant to the surgery. The computer system 110 can generate multiplanar reformatting slice projections 128 from the pre-operative imaging data, which entails reformatting the volumetric imaging data into two-dimensional slices along standardized anatomical planes including axial, sagittal, and coronal orientations. The multiplanar reformatting slice projections 128 can provide cross-sectional views of the anatomy that enable detailed examination of anatomical structures and their spatial relationships. The augmented reality display system 102 can display the multiplanar reformatting slice projections 128 as two-dimensional image planes positioned in three-dimensional space to facilitate surgical planning and navigation.

The computer system 110 can generate a skin surface hologram 130 based on the pre-operative imaging data. The skin surface hologram 130 can represent the external body surface of the patient derived from the tomographic imaging data. The augmented reality system 102 can display the skin surface hologram 130 with user-adjustable opacity settings. The adjustable opacity can enable operators to see through the skin surface hologram 130 to visualize the three-dimensional representations of anatomical structures 126 positioned beneath the surface. The operator can increase opacity to emphasize the skin surface location or decrease opacity to emphasize the internal anatomical structures, enabling simultaneous visualization of external entry points and internal anatomy.

The augmented reality system 102 can display the three-dimensional representations of anatomical structures 126 in spatial alignment with the skin surface hologram 130 and multiplanar reformatting slice projections 128. The operator can identify an anatomical target location within the three-dimensional representations of anatomical structures 126 where surgical access is required. The anatomical target can include a tumor, an organ, a vessel, or any other structure of surgical interest. The computer system 110 can generate an anatomical target marker 132 positioned at the identified anatomical target location. The anatomical target marker 132 can be displayed as a three-dimensional object including a sphere, crosshair, bullseye, or other visual indicator that clearly identifies the target location within the anatomical structures. The augmented reality system 102 can display the multiplanar reformatting slice projections 128 in coordination with the anatomical target marker 132. The computer system 110 can generate target intersection markers 134 that indicate where the anatomical target location intersects with each of the multiplanar reformatting slice projections 128. Each target intersection marker 134 can be displayed on the corresponding multiplanar reformatting slice projection 128, showing the position of the anatomical target within that particular imaging plane. The target intersection markers 134 can be rendered as crosshairs, circles, or other visual indicators overlaid on the multiplanar reformatting slice projections 128. The correlation between the three-dimensional anatomical target marker 132 and the two-dimensional target intersection markers 134 on multiple imaging planes can enable the operator to verify the target location across different anatomical views and confirm accurate target identification.

The augmented reality system 102 can enable cross-referencing between multiple imaging modalities to verify the anatomical target location. The operator can view the same anatomical location in CT data, MRI data, and PET data to confirm the target position. The anatomical target marker 132 can be displayed consistently across different imaging modalities, maintaining the same spatial location while the underlying imaging data changes. The target intersection markers 134 can be updated to reflect the target location on multiplanar reformatting slice projections 128 generated from each imaging modality. The cross-referencing capability can enable the operator to leverage the strengths of different imaging modalities, using CT for bone and dense tissue visualization, MRI for soft tissue contrast, and PET for metabolic activity assessment.

The augmented reality system 102 can enable selective visualization of specific anatomical structures during surgical planning and navigation. The operator can activate or deactivate display of particular organs, vessels, or bone structures. The augmented reality system 102 can display anatomical structures with adjustable transparency, enabling the operator to see through one structure to visualize structures behind it. For example, the augmented reality system 102 can display a stomach hologram with reduced transparency, enabling the operator to see deeper anatomical structures. The augmented reality system 102 can display bone holograms showing rib locations or pelvic structures. The operator can layer multiple anatomical structures with varying transparency settings to build a comprehensive understanding of the spatial relationships between different anatomical features.

The augmented reality system 102 can be configured to provide laparoscopic port placement planning capabilities using the three-dimensional representations of anatomical structures 126, multiplanar reformatting slice projections 128, skin surface hologram 130, and anatomical target marker 132. The augmented reality system 102 can enable placement of one or more virtual lap arms 136, each virtual lap arm 136 corresponding to a proposed laparoscopic instrument path. Each virtual lap arm 136 can include a proximal end 136a and a distal end. The proximal end 136a of each virtual lap arm 136 can be positioned at the anatomical target marker 132. The distal end of each virtual lap arm 136 can extend outward from the anatomical target to intersect with the skin surface hologram 130, defining a proposed entry point on the external body surface. Each virtual lap arm 136 can represent the complete path of a laparoscopic instrument 106 from the skin entry point through tissue to the anatomical target marker 132.

The augmented reality system 102 can automatically generate initial positions for multiple virtual lap arms 136, each corresponding to a proposed laparoscopic instrument path, based on predetermined port configurations. The computer system 110 can automatically locate the umbilicus on the skin surface hologram 130 using anatomical landmark detection. For a three-port configuration, the augmented reality system 102 can position a first virtual lap arm 136 through the umbilicus to serve as an optical port, with the proximal end 136a positioned at the anatomical target marker 132. The augmented reality system 102 can position additional working port virtual lap arms 136 at default distances and angles from the first virtual lap arm 136. The predetermined configuration can arrange the virtual lap arms 136 in geometric patterns including a baseball diamond configuration where the optical port position serves as home plate, the anatomical target marker 132 serves as second base, and working ports at first and third base positions. Other predetermined configurations can include semi-circular patterns or linear arrangements depending on the surgical procedure.

The augmented reality system 102 can display the virtual lap arms 136 overlaid with the three-dimensional representations of anatomical structures 126, skin surface hologram 130, and multiplanar reformatting slice projections 128. The virtual lap arms 136 can be rendered as three-dimensional objects with distinct colors or visual properties to distinguish them from anatomical structures. The augmented reality system 102 can display the proximal ends 136a of the virtual lap arms 136 clustered at the anatomical target marker 132 and the distal ends distributed across the skin surface hologram 130 at the proposed entry points. The visualization can enable the operator to assess the spatial arrangement of all proposed laparoscopic instrument paths simultaneously. The convergence of multiple virtual lap arms 136 at the anatomical target marker 132 can create a laparoscopic instrument trajectory intersection region 138 where the proposed insertion paths meet, providing visual confirmation that all planned ports will provide access to the same target location.

The computer system 110 can determine laparoscopic instrument trajectories 140 for each of the virtual lap arms 136. Each laparoscopic instrument trajectory 140 can represent the linear path extending along the longitudinal axis of the corresponding virtual lap arm 136 from the skin entry point to the anatomical target marker 132. The augmented reality system 102 can display the laparoscopic instrument trajectories 140 as lines, rays, or tubular projections that visually represent the insertion paths. The laparoscopic instrument trajectories 140 can be displayed with visual properties including color, transparency, and thickness to enhance visibility against the anatomical structures. The laparoscopic instrument trajectory intersection region 138 can be displayed where the laparoscopic instrument trajectories 140 converge at the anatomical target marker 132, visually indicating the common target point for all planned laparoscopic instrument insertion paths.

The augmented reality system 102 can enable interactive movement and adjustment of the virtual lap arms 136. The operator can select and grab the distal end of a virtual lap arm 136 using hand gestures, voice commands, or manual control interfaces. While the proximal end 136a of the virtual lap arm 136 remains anchored at the anatomical target marker 132, the operator can move the distal end along the skin surface hologram 130 to reposition the proposed entry point. As the distal end moves, the virtual lap arm 136 can pivot about the proximal end, changing the angle and trajectory of the proposed laparoscopic instrument insertion path. The augmented reality system 102 can automatically calculate the intersection point between the repositioned virtual lap arm 136 and the skin surface hologram 130 in real-time, continuously updating the entry point position as the operator adjusts the virtual lap arm 136.

During interactive adjustment, the augmented reality system 102 can provide real-time feedback. The augmented reality system 102 can display quantitative measurements including the distance from the skin entry point to the anatomical target marker 132 along each virtual lap arm 136. The augmented reality system 102 can display angular measurements between adjacent virtual lap arms 136, enabling the operator to maintain adequate spacing for instrument manipulation. The augmented reality system 102 can display the depth of insertion for each laparoscopic instrument path. The measurements can update dynamically as the operator moves the virtual lap arms 136, providing continuous feedback during the planning process.

The computer system 110 can perform collision detection analysis between the laparoscopic instrument trajectories 140 and the three-dimensional representations of anatomical structures 126. The computer system 110 can identify anatomical structures that intersect or come within a threshold distance of the laparoscopic instrument trajectories 140. Critical structures can include major vessels, organs, bones, or other anatomical features that should be avoided during laparoscopic instrument insertion. The augmented reality system 102 can detect when a virtual lap arm 136 is positioned too close to a critical structure and generate a warning. The warning can be visual, displaying the virtual lap arm 136 in a warning color such as red or yellow. The warning can be audio, generating an audible alert. The warning can be haptic, providing tactile feedback through a controller. The augmented reality system 102 can highlight the specific anatomical structure at risk and display the distance between the laparoscopic instrument trajectory 140 and the critical structure.

The computer system 110 can generate virtual port markers 142 at the locations where the distal ends of the virtual lap arms 136 intersect with the skin surface hologram 130. The virtual port markers 142 can be displayed as distinct visual indicators on the skin surface hologram 130, marking the precise entry points for laparoscopic instrument insertion. The virtual port markers 142 can be rendered as circles, crosses, targets, or other symbols that clearly indicate the insertion locations. The virtual port markers 142 can be automatically updated in real-time as the operator adjusts the positions of the virtual lap arms 136, maintaining continuous indication of the current planned entry points.

The augmented reality system 102 can display the multiplanar reformatting slice projections 128 during port placement planning to provide cross-sectional visualization of the laparoscopic instrument trajectories 140 through anatomical structures. The multiplanar reformatting slice projections 128 can show how the laparoscopic instrument trajectories 140 pass through tissue layers, organs, and vessels in axial, sagittal, and coronal planes. The target intersection markers 134 displayed on each multiplanar reformatting slice projection 128 can show where the anatomical target appears in each imaging plane, enabling correlation between the three-dimensional port placement visualization and two-dimensional cross-sectional imaging. The operator can verify that laparoscopic instrument trajectories 140 are appropriately aligned with the anatomical target marker 132 by confirming that the trajectories pass through the target intersection markers 134 on the corresponding multiplanar reformatting slice projections 128.

The computer system 110 can store port placement plans including the positions of the virtual lap arms 136, virtual port markers 142, anatomical target marker 132, and associated laparoscopic instrument trajectories 140. The stored port placement plan can include the three-dimensional coordinates of all entry points on the skin surface, the three-dimensional coordinates of the anatomical target, the angles and orientations of all laparoscopic instrument trajectories 140, the positions of target intersection markers 134 on multiplanar reformatting slice projections 128, and quantitative measurements including insertion depths and inter-port angles. The stored port placement plans can be retrieved during procedures. The augmented reality system 102 can display the stored port placement plan overlaid on the patient during the procedure, providing visual guidance for laparoscopic instrument insertion at the planned locations. The port placement planning can be performed pre-operatively, enabling advanced surgical planning before the patient enters the operating room, or intraoperatively, enabling real-time planning and adjustment based on current patient positioning and anatomy.

The system 100 can be configured to display ultrasound flashlight visualizations that project ultrasound imaging data in three-dimensional space aligned with the imaging plane of the ultrasound probe 108. The computer system 110 can receive ultrasound imaging data from the ultrasound probe 108 during laparoscopic procedures. The ultrasound imaging data can include two-dimensional cross-sectional images of internal anatomical structures captured by the ultrasound probe 108. The ultrasound imaging data can be transmitted continuously in real-time as the ultrasound probe 108 acquires images, enabling live visualization of anatomical structures during surgical manipulation.

The computer system 110 can generate ultrasound flashlight projections 144 by positioning the two-dimensional ultrasound imaging data at the calculated location and orientation of the imaging plane in three-dimensional space. The ultrasound flashlight projection 144 can display the ultrasound image as if it were projected onto the imaging plane itself, creating a visualization where the ultrasound data appears at its actual physical location within the patient anatomy. The ultrasound flashlight projection 144 can be scaled to match the physical dimensions of the imaging plane based on the ultrasound probe specifications and imaging depth settings.

The ultrasound flashlight projection 144 can be displayed in combination with the three-dimensional representations of anatomical structures 126 and multiplanar reformatting slice projections 128 derived from pre-operative imaging. The combination can enable the operator to correlate real-time intraoperative ultrasound imaging with pre-operative CT, MRI, or PET imaging. The operator can visualize how the two-dimensional ultrasound slice intersects with three-dimensional anatomical models and how it compares to the multiplanar reformatting slice projections 128 from pre-operative imaging, providing spatial context for the ultrasound imaging data. The ultrasound flashlight projection 144 can reveal tissue characteristics, vessel flow, or anatomical features that may have changed since pre-operative imaging or that are better visualized with ultrasound modality.

The ultrasound flashlight projection 144 can be displayed in combination with the laparoscopic instruments 106 and their holographic representations. The operator can visualize the spatial relationship between the ultrasound imaging plane and the positions of laparoscopic instruments, enabling coordinated manipulation where ultrasound guidance directs instrument positioning. The ultrasound flashlight projection 144 can display instruments that have entered the ultrasound imaging plane, showing the instrument tips within the ultrasound image while simultaneously showing the physical instruments and their holographic projections in the surrounding three-dimensional space.

The system 100 can be configured to display holographic light ray projections 146 that represent the trajectories of the laparoscopic instruments 106 as linear extensions projecting from the instruments into the surgical field. The holographic light ray projections 146 can provide visual guidance for instrument navigation by showing where an instrument is aimed and what anatomical structures lie along its path. The tracking system 104 can continuously track the position and orientation of each laparoscopic instrument 106 through the instrument sensors 120 attached via the mounting brackets 116. The computer system 110 can receive the tracking data indicating the three-dimensional position and three-dimensional orientation of each instrument sensor 120. The computer system 110 can retrieve the calibration data associated with each specific mounting bracket 116 and instrument sensor 120 combination. The calibration data can define the geometric relationship between the instrument sensor 120 and functional reference points on the laparoscopic instrument 106, including the instrument tip, instrument shaft axis, or tool working axis.

The computer system 110 can determine the instrument axis for each laparoscopic instrument 106 by applying the calibration data to the tracking data. The instrument axis can represent the longitudinal centerline of the instrument shaft or the functional working axis of the instrument tool. For linear instruments such as needles, trocars, or ablation probes, the instrument axis can align with the shaft and extend forward from the instrument tip. For articulated instruments, the instrument axis can represent the orientation of the end effector or tool component. The computer system 110 can calculate a three-dimensional line extending along the instrument axis, defining the trajectory the instrument is following or would follow if advanced forward.

The computer system 110 can generate holographic light ray projections 146 as visual representations extending along the calculated instrument axes. Each holographic light ray projection 146 can extend forward from the corresponding laparoscopic instrument 106, projecting into the surgical field to visualize the instrument trajectory. The holographic light ray projections 146 can be rendered as lines, rays, or tubular beams with visual properties including color, brightness, and transparency to enhance visibility. Different instruments can be assigned different colors or visual properties to distinguish multiple holographic light ray projections 146 when several instruments are tracked simultaneously.

The holographic light ray projections 146 can be displayed in combination with the three-dimensional representations of anatomical structures 126, multiplanar reformatting slice projections 128, and anatomical target marker 132. The combination can enable the operator to visualize the relationship between instrument trajectories and internal anatomy. The operator can see where the holographic light ray projection 146 intersects with organs, vessels, tumors, or other anatomical targets. The operator can visualize how the holographic light ray projection 146 aligns with the anatomical target marker 132 and passes through the target intersection markers 134 on the multiplanar reformatting slice projections 128. The visualization can guide instrument positioning by showing whether the instrument trajectory will reach the intended target and what structures lie along the approach path.

The computer system 110 can perform intersection analysis between the holographic light ray projections 146 and the three-dimensional representations of anatomical structures 126. The computer system 110 can determine whether a holographic light ray projection 146 intersects with an anatomical structure of interest and calculate the three-dimensional coordinates of the intersection point. For target structures such as tumors or lesions marked by the anatomical target marker 132, the computer system 110 can determine when the holographic light ray projection 146 is aligned with the target, indicating that advancing the laparoscopic instrument 106 would reach the target location. The augmented reality system 102 can provide visual feedback indicating target alignment, such as highlighting the anatomical target marker 132 or displaying the holographic light ray projection 146 in a confirmation color when proper alignment is achieved.

The computer system 110 can determine distances between the laparoscopic instrument 106 and anatomical structures along the holographic light ray projection 146. The computer system 110 can calculate the distance from the current instrument tip position to an intersection point with a target structure marked by the anatomical target marker 132. The augmented reality system 102 can display distance indicators along the holographic light ray projection 146, showing how far the instrument must be advanced to reach the target. The distance indicators can update in real-time as the operator advances or retracts the instrument, providing continuous feedback on instrument depth.

The holographic light ray projections 146 can provide guidance for specific procedural tasks. For needle placement procedures, the holographic light ray projection 146 can show the needle trajectory toward a biopsy target or injection site marked by the anatomical target marker 132. For insertion of a laparoscopic instrument 106 during port placement, the holographic light ray projection 146 can confirm that the laparoscopic instrument is aligned with the planned laparoscopic instrument trajectory 140 from the port placement plan, showing alignment with the laparoscopic instrument trajectory intersection region 138 at the anatomical target marker 132. For ablation probe positioning, the holographic light ray projection 146 can guide the ablation probe tip to the center of a tumor or lesion while showing surrounding critical structures to avoid. For vessel navigation, the holographic light ray projection 146 can show the relationship between instrument trajectory and vascular structures, enabling the operator to navigate along vessels or avoid vessel injury.

The computer system 110 can detect potential collisions between holographic light ray projections 146 and critical anatomical structures. When a holographic light ray projection 146 is aimed toward a critical structure that should be avoided, the augmented reality system 102 can provide collision warnings. The collision warnings can be visual, displaying the holographic light ray projection 146 in a warning color or highlighting the at-risk anatomical structure. The collision warnings can be audio alerts. The collision warnings can indicate the distance between the instrument trajectory and the critical structure. The operator can adjust the instrument position or orientation in response to collision warnings to redirect the instrument trajectory away from critical structures.

The computer system 110 can generate holographic representations 148 corresponding to the tracked laparoscopic instruments 106 and ultrasound probe 108. The holographic representations 148 can be three-dimensional virtual models of the laparoscopic instruments 106 and ultrasound probe 108 that match the physical devices in shape and size. The holographic representations 148 can display the instruments with enhanced visibility, distinctive colors, or visual highlighting to distinguish them from the physical instruments and anatomical structures. The augmented reality system 102 can display the holographic representations 148 positioned and oriented to align with the corresponding physical laparoscopic instruments 106 and ultrasound probe 108, creating augmented views where virtual instrument models overlay or complement the physical instruments.

The system 100 can be configured to provide real-time surgical navigation throughout laparoscopic procedures by integrating tracking data, holographic visualizations, multiplanar reformatting slice projections 128, ultrasound flashlight projections 144, holographic light ray projections 146, and pre-operative imaging into a comprehensive guidance system. The tracking system 104 can continuously track the positions and orientations of all laparoscopic instruments 106 and the ultrasound probe 108 through the instrument sensors 120. The tracking system 104 can transmit tracking data to the computer system 110 at high frequency, providing real-time updates of instrument positions. The computer system 110 can apply the calibration data for each mounting bracket 116 and instrument sensor 120 combination to determine the positions and orientations of the functional portions of the instruments, including instrument tips, shaft axes, and imaging planes. Surgical navigation can enable the operator to visualize instrument positions relative to anatomical structures, navigate to target locations marked by anatomical target markers 132, avoid critical structures, and perform surgical tasks with enhanced spatial awareness.

The augmented reality system 102 can be configured to operate in two distinct visualization modes: an in-situ projection mode and a float projection mode. The augmented reality system 102 can support transitions between these modes while continuously maintaining accurate tracking and registration of all surgical instruments and imaging data throughout the procedure. The relationship between holographic light ray projections 146, ultrasound flashlight projections 144, multiplanar reformatting slice projections 128, and three-dimensional representations of anatomical structures 126 can be maintained to facilitate the operative task and associated decision-making.

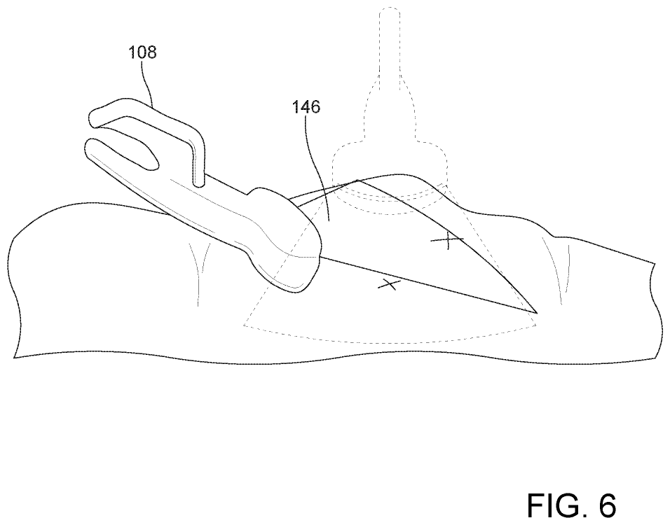

With reference to FIG. 6, in the in-situ projection mode, the augmented reality system 102 can be configured to enable registration and projection of holographic elements in direct alignment with corresponding physical devices and anatomical targets on the body of the patient. The augmented reality system 102 can project the ultrasound flashlight projections 144, holographic light ray projections 146, and holographic representations 148 directly onto corresponding physical devices and target tissue. Through the tracking system 104, the holographic projections can maintain continuous spatial alignment with the laparoscopic instruments 106 and ultrasound probe 108 throughout the procedure. The in-situ projection mode can provide immediate visual feedback for surgical navigation while maintaining direct correlation between physical devices and their holographic representations. The in-situ projection mode can facilitate correlation between hand manipulation of the laparoscopic instruments 106 and the holographic guidance. The augmented reality system 102 can enable voice command control of holographic elements in the in-situ projection mode, allowing handsfree adjustment of visualization parameters and holographic display settings during the procedure. Each holographic light ray projection 146 can appear to extend directly from the corresponding physical instrument, and each ultrasound flashlight projection 144 can appear to emanate from the physical ultrasound probe 108, positioned at the actual imaging plane location within the patient's body.

In the float projection mode, the augmented reality system 102 can be configured to position holographic projections away from direct patient alignment while maintaining geometric relationships between the tracked laparoscopic instruments 106, ultrasound probe 108, and all visualized elements through application of a consistent rigid affine transform. The float projection mode can enable adjustment of projections relative to the background scene, mitigate against problematic viewing angles of the ultrasound imaging plane, and allow modification relative to the laparoscopic camera view. The augmented reality system 102 can support translation and scaling without rotation to optimize visualization and eye-hand movement correlation. The float projection can be translated for side-by-side visualization with the optical laparoscopic display while maintaining an orientation to facilitate eye-hand movement correlation. In float projection mode, the augmented reality system 102 can display multiplanar reformatting slice projections 128, ultrasound flashlight projections 144, holographic light ray projections 146, three-dimensional representations of anatomical structures 126, anatomical target markers 132, and holographic representations of instruments 148 positioned in three-dimensional space to provide comprehensive surgical visualization away from the patient's body.

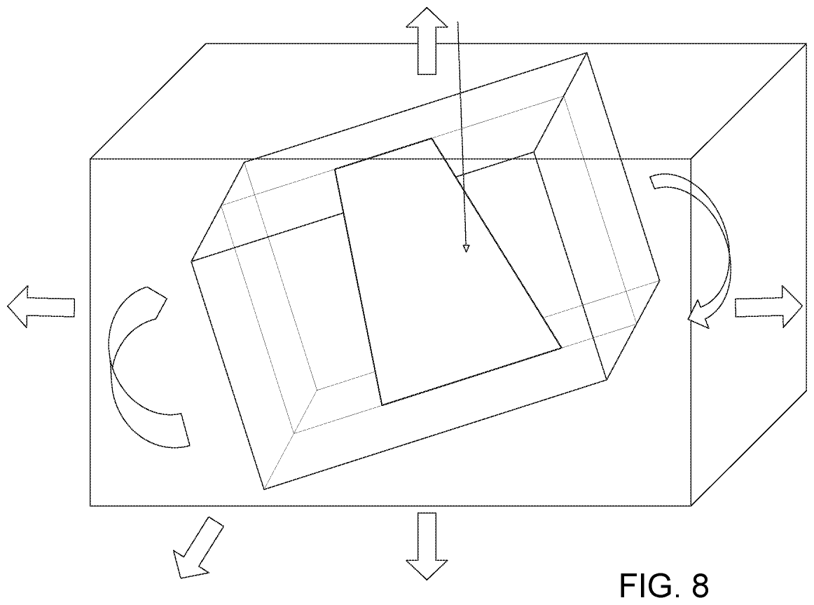

With reference to FIG. 8, to maintain geometric accuracy in float projection mode, the augmented reality system 102 can apply the same rigid affine transform to all tracked devices and imaging projections, enabling consistent spatial relationships between the ultrasound flashlight projections 144, multiplanar reformatting slice projections 128, holographic light ray projections 146, holographic representations 148 of the laparoscopic instruments 106, and three-dimensional representations of anatomical structures 126 including the anatomical target marker 132. The augmented reality system 102 can preserve target registration error while providing controls for rotation and scaling that maintain device relationships. When the operator adjusts the position or orientation of float mode visualizations, all elements move together as a unified group, maintaining their spatial relationships.

The augmented reality system 102 can be configured with bounding box capabilities to enable controlled manipulation of holographic projections through multiple rotational axes during float projection mode. The bounding box can be a three-dimensional interactive control interface displayed around selected holographic elements, providing visual handles and control points for manipulation of the holographic projections. The augmented reality system 102 can provide pitch control for up/down rotation, yaw control for left/right rotation, and roll control about the normal vector of the ultrasound flashlight projection 144 or selected multiplanar reformatting slice projection 128. The bounding box interface can enable “Rotate Toward” commands that can orient the ultrasound flashlight projection 144 or multiplanar reformatting slice projections 128 toward the operator's view direction. The augmented reality system 102 can support “Rotate Clockwise/Counterclockwise” controls about the normal vector to the ultrasound flashlight projection 144 or multiplanar reformatting slice projections 128. The augmented reality system 102 can utilize far and near interaction handles for three-axis rotation. The handles can enable control over pitch, yaw, and roll adjustments while maintaining geometric relationships between tracked devices through application of the same rigid transform to all tracked devices.

The augmented reality system 102 can address problematic viewing angles of the ultrasound flashlight projection 144 and multiplanar reformatting slice projections 128 through float projection mode capabilities. When an imaging plane is oriented nearly edge-wise toward the operator's viewpoint, the ultrasound flashlight projection 144 or multiplanar reformatting slice projection 128 can appear as a thin line that is difficult to interpret. The augmented reality system 102 can position the ultrasound flashlight projection 144 or multiplanar reformatting slice projections 128 away from direct patient alignment and apply rotational adjustments to present the imaging planes at optimal viewing angles. The operator can issue voice commands including “Rotate Toward” to orient the ultrasound flashlight projection 144 or selected multiplanar reformatting slice projection 128 approximately toward the operator's view direction, ensuring the ultrasound image or reformatted slice is displayed face-on rather than edge-wise.

The augmented reality system 102 can provide control mechanisms in float projection mode including voice commands for adjustments. Voice commands can include “rotate toward” commands that can orient the ultrasound flashlight projection 144 or multiplanar reformatting slice projections 128 toward the view of the operator. Voice commands can include “rotate clockwise/counterclockwise” controls about the normal vector of the ultrasound flashlight projection 144 or selected multiplanar reformatting slice projection 128. The augmented reality system 102 can provide a “Reset Float” command for return to in-situ projection mode. During rotation adjustments, the augmented reality system 102 can notify operators when rotation is changed by more than a predetermined number of degrees on any of the three axes.

The augmented reality system 102 can support port placement planning in both in-situ projection mode and float projection mode. In in-situ projection mode, the augmented reality system 102 can display the skin surface hologram 130, virtual lap arms 136, laparoscopic instrument trajectories 140, virtual port markers 142, and anatomical target marker 132 directly aligned with the physical patient's body. In float projection mode, the augmented reality system 102 can position the entire port planning visualization away from the patient's body while maintaining the geometric relationships between all elements. The multiplanar reformatting slice projections 128 can be particularly advantageous in float projection mode, enabling the operator to view cross-sectional imaging planes positioned in three-dimensional space alongside the three-dimensional anatomical structures 126, anatomical target marker 132, virtual lap arms 136, and laparoscopic instrument trajectories 140. The target intersection markers 134 can be displayed on the multiplanar reformatting slice projections 128 in float mode, showing where the anatomical target location appears on each imaging plane and facilitating correlation between two-dimensional slice views and three-dimensional spatial planning. Float projection mode can enable port placement planning without requiring direct projection onto the patient, accommodating factors including respiratory motion, changes in abdominal shape due to insufflation, adjustments to operating table position, and variations in digestive state of the patient. The operator can mentally correlate the floating port plan with the patient's anatomy while avoiding the technical challenges of maintaining precise millimeter-level registration during dynamic conditions.

The augmented reality system 102 can be configured to account for patient table adjustments during laparoscopic procedures. The position of the patient table can be adjusted including tilt, which can cause tracked devices to shift their position and orientation relative to their corresponding projected holograms. When utilizing a table top generator (TTG) as the field generator of the tracking system 104, the augmented reality system 102 can maintain approximate alignment during table adjustments. The table top generator can move simultaneously with the patient table, patient, and surgical devices. This simultaneous movement can result in minimal changes to tracking position and orientation of the devices, allowing their holographic projections to remain approximately consistent and stationary. If misalignment occurs, the augmented reality system 102 can enable re-registration to re-align co-projection of the ultrasound flashlight projections 144, holographic light ray projections 146, and other holographic elements with their corresponding physical entities. The augmented reality system 102 can provide a reset float command for return to in-situ projection mode while maintaining continuous updates from the tracking system 104 to preserve spatial relationships between tracked devices and their holographic representations.

When implementing a planar field generator (PFG) as the field generator that is not attached to the patient table, the augmented reality system 102 can require additional registration capabilities. Since the planar field generator can remain stationary while the surgical table moves, adjusting the table position will not simultaneously adjust the patient and tracked devices with similar movement. The tracking position and orientation of the laparoscopic instruments 106 and ultrasound probe 108 can update due to the table movement, which can cause misalignment between projections and corresponding physical devices and anatomy. Upon completion of the table movement, the augmented reality system 102 can enable re-registration to re-align co-projection of the ultrasound flashlight projections 144, holographic light ray projections 146, and other holographic elements with corresponding physical entities.

The augmented reality system 102 can be configured to provide laparoscopic surgical guidance using pre-operative tomographic imaging integrated with real-time tracking and holographic visualization. The computer system 110 can receive pre-operative imaging data from the imaging systems 112. The pre-operative imaging data can include Computed Tomography (CT) scans, Magnetic Resonance Imaging (MRI) scans, Positron Emission Tomography (PET) scans, or other tomographic imaging data. The computer system 110 can process the pre-operative imaging data to generate the three-dimensional representations of anatomical structures 126 including organs, vessels, bones, tumors, and other structures relevant to the surgery.

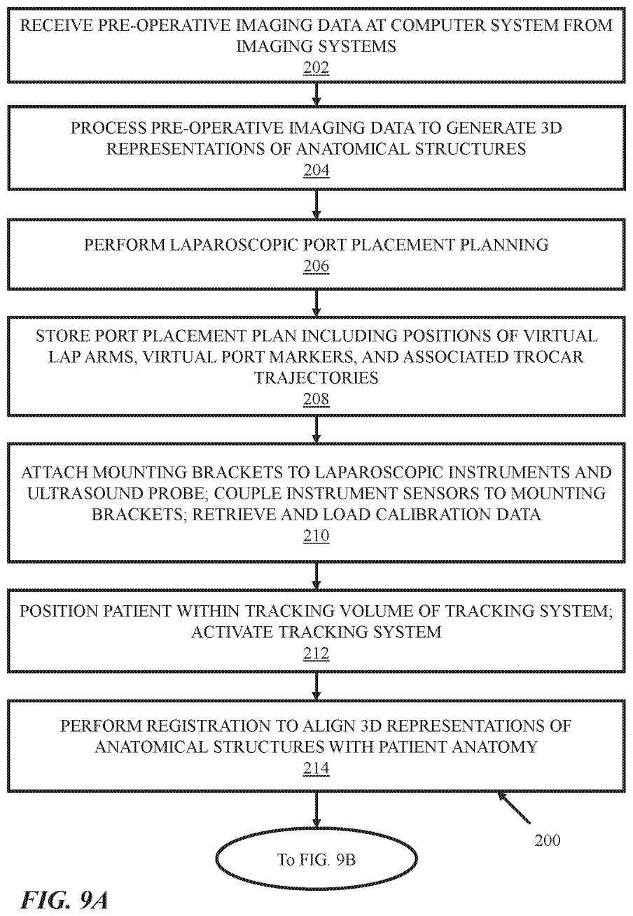

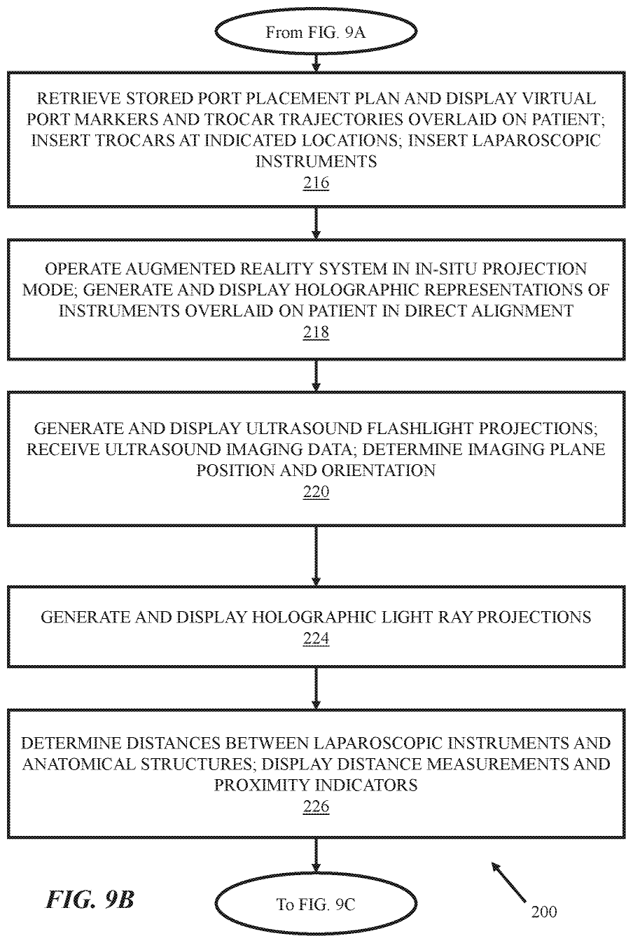

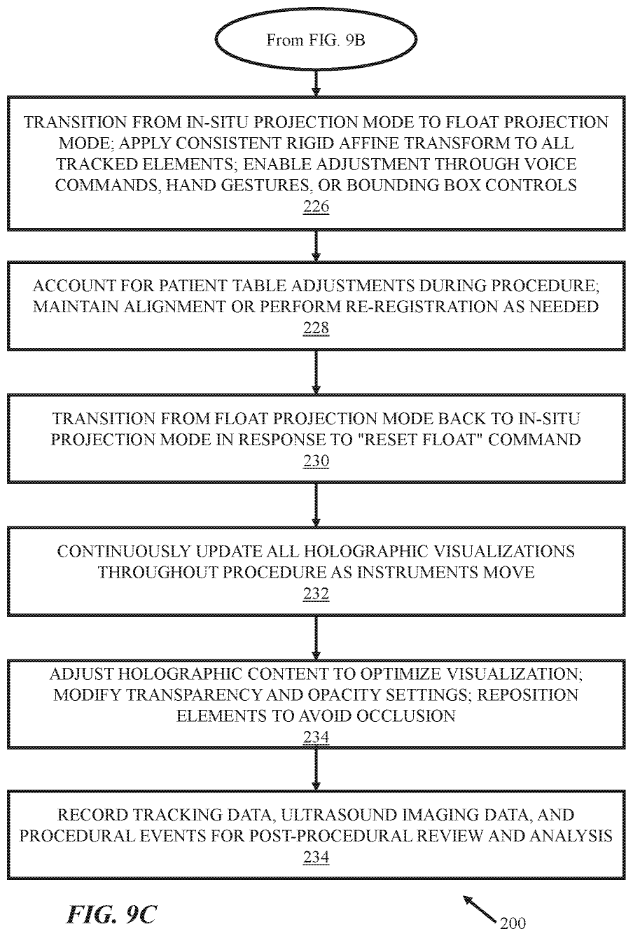

A method 200 of providing augmented reality guidance for laparoscopic procedures can be performed using the system 100, for example, as depicted in FIGS. 9A-9C. The method 200 can include a step 202 of receiving pre-operative imaging data at the computer system 110 from the imaging systems 112. The pre-operative imaging data can include Computed Tomography (CT) scans, Magnetic Resonance Imaging (MRI) scans, Positron Emission Tomography (PET) scans, or other tomographic imaging data of the patient.

The method 200 can include a step 204 of processing the pre-operative imaging data to generate the three-dimensional representations of anatomical structures 126. The computer system 110 can segment anatomical structures from the pre-operative imaging data including organs, vessels, bones, tumors, and other structures relevant to the surgery. The computer system 110 can generate the skin surface hologram 130 representing the external body surface of the patient. The computer system 110 can generate multiplanar reformatting slice projections 128 from the pre-operative imaging data, reformatting the volumetric imaging data into two-dimensional slices along standardized anatomical planes including axial, sagittal, and coronal orientations.

The method 200 can include a step 206 of performing laparoscopic port placement planning. The step 206 can include displaying the three-dimensional representations of anatomical structures 126, multiplanar reformatting slice projections 128, and the skin surface hologram 130 through the augmented reality system 102. The step 206 can include identifying an anatomical target location within the three-dimensional representations of anatomical structures 126. The step 206 can include generating an anatomical target marker 132 positioned at the anatomical target location. The step 206 can include generating target intersection markers 134 that indicate where the anatomical target location intersects with each of the multiplanar reformatting slice projections 128. The step 206 can include automatically generating initial positions for multiple virtual lap arms 136 based on predetermined port configurations, including automatically locating the umbilicus on the skin surface hologram 130 and positioning virtual lap arms 136 in geometric arrangements such as a baseball diamond configuration with the anatomical target marker 132 serving as a central reference point.

The step 206 can include enabling interactive adjustment of the virtual lap arms 136 by allowing an operator to grab and move the distal end of each virtual lap arm 136 while the proximal end 136a remains anchored at the anatomical target marker 132. The step 206 can include automatically calculating and updating virtual port locations by determining the intersection with the skin surface hologram 130 in real-time as the virtual lap arms 136 are adjusted. The step 206 can include determining laparoscopic instrument trajectories 140 for each of the virtual lap arms 136, with the laparoscopic instrument trajectories 140 converging at the anatomical target marker 132 to create a laparoscopic instrument trajectory intersection region 138. The step 206 can include performing collision detection to identify intersections between the laparoscopic instrument trajectories 140 and critical anatomical structures. The step 206 can include generating and displaying warnings if a virtual lap arm 136 is too close to a critical structure. The step 206 can include generating virtual port markers 142 at the locations where the virtual lap arms 136 intersect with the skin surface hologram 130. The step 206 can include verifying laparoscopic instrument trajectory alignment by confirming that the laparoscopic instrument trajectories 140 pass through the target intersection markers 134 on the corresponding multiplanar reformatting slice projections 128.

The method 200 can include a step 208 of storing a port placement plan including the positions of the virtual lap arms 136, virtual port markers 142, anatomical target marker 132, associated laparoscopic instrument trajectories 140, and positions of target intersection markers 134 on multiplanar reformatting slice projections 128 in the computer system 110. The port placement planning of step 206 can be performed pre-operatively or intraoperatively.

The method 200 can include a step 210 of attaching the mounting brackets 116 to the laparoscopic instruments 106 and ultrasound probe 108 that will be used during the procedure. The step 210 can include coupling the instrument sensors 120 to the mounting brackets 116. The step 210 can include retrieving calibration data associated with each mounting bracket 116 and instrument sensor 120 combination and loading the calibration data into the computer system 110. The method 200 can include a step 212 of positioning the patient within the tracking volume of the tracking system 104. The step 212 can include activating the tracking system 104 to begin tracking the instrument sensors 120 attached to the laparoscopic instruments 106 and ultrasound probe 108. The method 200 can include a step 214 of performing registration to align the three-dimensional representations of anatomical structures 126 with the patient anatomy. The step 214 can include identifying anatomical landmarks on the patient and corresponding landmarks in the pre-operative imaging data, or placing fiducial markers on the patient and identifying the fiducial markers in both the physical environment and the imaging data.

The method 200 can include a step 216 of retrieving the stored port placement plan and displaying the virtual port markers 142, laparoscopic instrument trajectories 140, and anatomical target marker 132 overlaid on the patient at the planned insertion locations through the augmented reality system 102. The step 216 can include displaying the laparoscopic instrument trajectory intersection region 138 where the laparoscopic instrument trajectories 140 converge at the anatomical target marker 132. The step 216 can include inserting trocars at the locations indicated by the virtual port markers 142 using the displayed laparoscopic instrument trajectories 140 as guidance. The step 216 can include inserting the laparoscopic instruments 106 through the trocar ports.

The method 200 can include a step 218 of operating the augmented reality system 102 in in-situ projection mode during the laparoscopic procedure. The step 218 can include continuously receiving tracking data from the tracking system 104 indicating the positions and orientations of the laparoscopic instruments 106 and ultrasound probe 108. The step 218 can include generating holographic representations of instruments 148 corresponding to the tracked laparoscopic instruments 106 and ultrasound probe 108. The step 218 can include displaying the holographic representations of instruments 148 overlaid on the patient in direct alignment with the corresponding physical instruments.

The step 218 can include displaying the three-dimensional representations of anatomical structures 126, multiplanar reformatting slice projections 128, and anatomical target marker 132 registered to the patient anatomy in combination with the holographic representations of instruments 148, enabling the operator to visualize internal anatomy in spatial context relative to the tracked instruments. The step 218 can include projecting holographic content directly onto corresponding physical devices and anatomical targets on the body of the patient to facilitate eye-hand movement coordination during surgical navigation.

The method 200 can include a step 220 of generating and displaying ultrasound flashlight projections 144 during the procedure. The step 220 can include receiving ultrasound imaging data from the ultrasound probe 108. The step 220 can include determining the position and orientation of the imaging plane of the ultrasound probe 108 based on the tracking data and the calibration data associated with the ultrasound probe 108. The step 220 can include generating the ultrasound flashlight projections 144 by positioning the ultrasound imaging data in three-dimensional space corresponding to the imaging plane location and orientation. The step 220 can include displaying the ultrasound flashlight projections 144 overlaid on the patient, enabling visualization of ultrasound imaging data in spatial context relative to external anatomy, internal anatomical structures, multiplanar reformatting slice projections 128, and other surgical instruments. The step 220 can include continuously updating the ultrasound flashlight projections 144 as the ultrasound probe 108 moves to maintain alignment with the imaging plane.

The method 200 can include a step 222 of generating and displaying holographic light ray projections 146 during the procedure. The step 222 can include determining instrument axes for the laparoscopic instruments 106 based on the tracking data and calibration data. The step 222 can include generating the holographic light ray projections 146 extending along the instrument axes to visually represent instrument trajectories. The step 222 can include displaying the holographic light ray projections 146 aligned with the laparoscopic instruments 106, extending from the instruments into the surgical field. The step 222 can include continuously updating the holographic light ray projections 146 as the laparoscopic instruments 106 move to maintain alignment with instrument trajectories. The step 222 can include performing intersection analysis between the holographic light ray projections 146 and the three-dimensional representations of anatomical structures 126 to determine alignment with target structures marked by the anatomical target marker 132 or potential collisions with critical structures. The step 222 can include displaying visual feedback indicating target alignment when the holographic light ray projection 146 aligns with the anatomical target marker 132 or collision warnings when approaching critical structures. The step 222 can include determining distances from instrument tips to anatomical structures along the holographic light ray projections 146 and displaying distance indicators. The step 222 can include visualizing how the holographic light ray projections 146 pass through the target intersection markers 134 on the multiplanar reformatting slice projections 128 to verify alignment with the planned target.

The method 200 can include a step 224 of determining distances between the laparoscopic instruments 106 and anatomical structures of interest and displaying distance measurements providing quantitative feedback. The step 224 can include displaying proximity indicators that change visual properties based on distance thresholds, providing visual alerts when instruments approach critical structures.

The method 200 can include a step 226 of transitioning from in-situ projection mode to float projection mode. The step 226 can include positioning holographic projections away from direct patient alignment while maintaining geometric relationships between tracked instruments through application of a consistent rigid affine transform. The step 226 can include applying the same rigid affine transform to the holographic representations of instruments 148, ultrasound flashlight projections 144, holographic light ray projections 146, multiplanar reformatting slice projections 128, and three-dimensional representations of anatomical structures 126 including the anatomical target marker 132 to maintain accurate spatial relationships.

The step 226 can include translating the holographic content to a position such as eye level or beside the patient. The step 226 can include enabling adjustment of the floating holographic content through voice commands, hand gestures, or bounding box controls. The step 226 can include responding to “Rotate Toward” voice commands by orienting the ultrasound flashlight projections 144 or selected multiplanar reformatting slice projection 128 toward the operator's view direction. The step 226 can include responding to “Rotate Clockwise/Counterclockwise” voice commands by rotating the holographic content about the normal vector of the ultrasound flashlight projection 144 or selected multiplanar reformatting slice projection 128. The step 226 can include enabling manipulation of bounding box handles to perform pitch, yaw, and roll adjustments. The step 226 can include displaying the target intersection markers 134 on the multiplanar reformatting slice projections 128 in float mode to show where the anatomical target location appears on each imaging plane.

The method 200 can include a step 228 of accounting for patient table adjustments during the procedure. When a table top generator is used as the field generator 120, the step 228 can include maintaining approximate alignment as the table top generator moves with the patient table. When a planar field generator is used as the field generator 120, the step 228 can include detecting misalignment due to table movement and performing re-registration to re-align holographic projections with corresponding physical devices and anatomy.

The method 200 can include a step 230 of transitioning from float projection mode back to in-situ projection mode. The step 230 can include responding to a “Reset Float” voice command by returning holographic projections to direct alignment with physical devices and patient anatomy. The method 200 can include a step 232 of continuously updating all holographic visualizations throughout the procedure as the laparoscopic instruments 106 and ultrasound probe 108 move. The step 232 can include maintaining accurate visualization despite instrument motion, patient motion, or changes in surgical field configuration. The method 200 can include a step 234 of adjusting holographic content during the procedure to optimize visualization. The step 234 can include modifying transparency of the three-dimensional representations of anatomical structures 126 to enable viewing of underlying structures. The step 234 can include modifying opacity of the skin surface hologram 130. The step 234 can include adjusting visibility of multiplanar reformatting slice projections 128. The step 234 can include repositioning holographic elements to avoid occlusion of the surgical field. The step 234 can include maintaining spatial registration between holographic content and physical devices as visualization parameters are adjusted.

The method 200 can include a step 236 of recording tracking data, ultrasound imaging data, and procedural events during the procedure for post-procedural review and analysis. The step 236 can include storing procedural data including recorded tracking data, ultrasound imaging data, port placement plans, positions of anatomical target markers 132, and annotations or markers placed during the procedure upon completion of the procedure.

### EXAMPLES

### Example: Procedure-Specific Port Configurations and Collaborative Planning

The augmented reality system 102 can be implemented for various surgical procedures with procedure-specific port configurations. The augmented reality system 102 can support three-port, four-port, and six-port arrangements depending on procedural requirements and surgical approach. For standard laparoscopic procedures, the augmented reality system 102 implements a three-port baseball diamond configuration. The surgeon uses voice commands to selectively visualize anatomical structures during port planning. The surgeon states “show bone” and the augmented reality system 102 displays rib holograms within the three-dimensional representations of anatomical structures 126, enabling verification that virtual port markers 142 are positioned in intercostal spaces. The bone hologram display complements traditional palpation techniques and enables visualization of deeper structures that cannot be palpated. The surgeon states “show stomach” and the augmented reality system 102 displays the stomach hologram with adjustable transparency, allowing verification that laparoscopic instrument trajectories 140 do not intersect with the stomach wall.

For robotic procedures using the Da Vinci surgical system, as illustrated in FIG. 6B, the augmented reality system 102 adapts port placement to accommodate robotic arm geometry. The augmented reality system 102 increases the default spacing between virtual lap arms 128 to account for the larger working envelope required by robotic instruments compared to manual laparoscopic instruments. The augmented reality system 102 positions virtual lap arms 128 at angles that maintain optimal triangulation while preventing robotic arm collision during instrument manipulation. The system accounts for the additional assistant port required for robotic procedures, generating a four-port configuration with appropriate spacing.

For gynecological procedures, the augmented reality system 102 optimizes port placement considering the anatomical differences of the female pelvis. The computer system 110 displays pelvic anatomical structures including the uterus, ovaries, bladder, and major pelvic vessels within the three-dimensional representations of anatomical structures 136. The augmented reality system 102 positions virtual lap arms 128 to provide access to target anatomy while avoiding critical pelvic structures. The system enables visualization of the uterine fundus and adnexal structures to guide port placement for procedures such as hysterectomy or ovarian surgery.

For kidney procedures, the augmented reality system 102 facilitates both transperitoneal and retroperitoneal access planning. For retroperitoneal approaches, the computer system 110 displays the anatomical relationships between the kidney, psoas muscle, colon, and surrounding organs. The augmented reality system 102 enables port placement that accesses the retroperitoneal space while avoiding peritoneal entry. The surgeon can position virtual lap arms 128 to approach the kidney from posterior or lateral angles appropriate for nephrectomy or partial nephrectomy procedures.

The augmented reality system 102 can enable collaborative port planning for procedures involving multiple surgeons. Two surgeons wearing separate augmented reality headsets can simultaneously visualize and interact with the same port planning display. Each surgeon can grab and adjust different virtual lap arms 128 while the augmented reality system 102 maintains synchronized display across both headsets. The computer system 110 maintains accurate geometric relationships between all virtual lap arms 128 as multiple users interactively adjust port positions. The collaborative planning enables surgical teams to jointly optimize port placement, with experienced surgeons guiding trainees through optimal port selection while both users visualize the same holographic content.

The augmented reality system 102 can account for anatomical variations between pre-operative imaging and intraoperative conditions. During pre-operative port planning, the three-dimensional representations of anatomical structures 136 are generated from imaging acquired with the patient in a supine position without pneumoperitoneum. During the actual procedure, abdominal insufflation creates pneumoperitoneum, increasing intra-abdominal volume and altering anatomical relationships. The computer system 110 can apply deformation algorithms to adjust the three-dimensional representations of anatomical structures 136 to reflect the increased working space and altered anatomical positions during pneumoperitoneum. The augmented reality system 102 can display both the pre-insufflation anatomical configuration and the estimated post-insufflation configuration, enabling the surgeon to plan port placement accounting for the anatomical changes that will occur after insufflation. The system enables visualization of how organs such as the liver or spleen may shift position after pneumoperitoneum establishment, informing port placement decisions to maintain optimal access throughout the procedure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An augmented reality system for laparoscopic procedures, comprising:
a tracking system configured to track positions and orientations of a laparoscopic instrument and an ultrasound probe during a laparoscopic procedure;
a computer system communicatively coupled to the tracking system and configured to:
receive pre-operative imaging data from an imaging system;
generate a three-dimensional representation of an anatomical structure based on the pre-operative imaging data;
receive tracking data from the tracking system indicating the position and orientation of the laparoscopic instrument and the ultrasound probe;
receive ultrasound imaging data from the ultrasound probe;
determine an imaging plane position and orientation of the ultrasound probe based on the tracking data and calibration data associated with the ultrasound probe;
generate an ultrasound flashlight projection displaying the ultrasound imaging data positioned in three-dimensional space corresponding to the imaging plane; and
generate a holographic light ray projection representing a trajectory of the laparoscopic instrument based on the tracking data and calibration data associated with the laparoscopic instrument; and
an augmented reality display system communicatively coupled to the computer system and configured to display a holographic ultrasound flashlight projection, a holographic light ray projection, and a holographic three-dimensional representation of the anatomical structure.

2. The system of claim 1, wherein the computer system is further configured to generate a skin surface hologram representing an external body surface of a patient based on the pre-operative imaging data.

3. The system of claim 2, wherein the computer system is further configured to:
enable placement of a plurality of virtual lap arms, each one of the virtual lap arms defining a proposed laparoscopic instrument insertion path, each one of the virtual lap arms including a proximal end positioned at an anatomical target and a distal end extending to intersect with the skin surface hologram;
determine a laparoscopic instrument trajectory for each one of the virtual lap arms;
generate a virtual port marker at an intersection point where each one of the virtual lap arms meets the skin surface hologram; and
enable interactive adjustment of the position of each of the virtual lap arms.

4. The system of claim 3, wherein the computer system is configured to perform collision detection between the laparoscopic instrument trajectories and the three-dimensional representations of anatomical structures to identify intersections with critical anatomical structures.

5. The system of claim 3, wherein the computer system is configured to:
enable interactive adjustment of each of the virtual lap arms by allowing a user to move the distal end of each virtual lap arm along the skin surface hologram while the proximal end remains anchored at the anatomical target; and
automatically calculate and update each of the virtual port locations by determining intersections with the skin surface hologram in real-time as the virtual lap arms are adjusted.

6. The system of claim 1, wherein the augmented reality display system is configured to operate in an in-situ projection mode in which holographic elements are displayed in direct alignment with corresponding physical devices and anatomical targets on a body of a patient.

7. The system of claim 6, wherein the augmented reality display system is configured to operate in a float projection mode in which the ultrasound flashlight projection, the holographic light ray projection, the three-dimensional representation of the anatomical structure, and holographic representations of tracked instruments are positioned away from direct patient alignment while maintaining geometric relationships between tracked elements.

8. The system of claim 7, wherein the augmented reality display system is configured to enable adjustment of the holographic projections in float projection mode through at least one of a voice command, a hand gesture, or a bounding box control.

9. The system of claim 1, wherein the computer system is configured to:
generate a holographic representation of an instrument corresponding to each of the tracked laparoscopic instrument and the ultrasound probe; and
wherein the augmented reality display system is configured to display the holographic representations of instruments overlaid on a patient in alignment with corresponding the tracked laparoscopic instrument and the ultrasound probe.

10. The system of claim 1, wherein the computer system is configured to:
perform intersection analysis between the holographic light ray projection and the three-dimensional representation of the anatomical structure;
determine a distance measurement between the laparoscopic instrument and the anatomical structure along the holographic light ray projection; and
wherein the augmented reality display system is configured to display the distance measurement and a proximity indicator.

11. The system of claim 1, further comprising:
a plurality of mounting brackets configured to attach to each one of the laparoscopic instrument and the ultrasound probe; and
a plurality of instrument sensors, each one of the instrument sensors configured to couple to one of the mounting brackets and communicatively couple to the tracking system.

12. The system of claim 11, wherein the computer system stores calibration data defining geometric relationships between each instrument sensor and a functional reference point on the corresponding instrument.

13. A method of augmented reality guided port placement planning for laparoscopic procedures, comprising:
receiving pre-operative imaging data from an imaging system at a computer system;
processing the pre-operative imaging data to generate a three-dimensional representation of an anatomical structure and a skin surface hologram representing an external body surface of a patient;
displaying, through an augmented reality display system, the three-dimensional representation of the anatomical structure and the skin surface hologram;
identifying an anatomical target location within the three-dimensional representation of the anatomical structure;
generating a plurality of virtual lap arms, each virtual lap arm defining a proposed laparoscopic instrument insertion path, each virtual lap arm including a proximal end positioned at the anatomical target and a distal end extending to intersect with the skin surface hologram;
determining laparoscopic instrument trajectories for each of the virtual lap arms;
generating a plurality of virtual port markers at intersection points where the virtual lap arms meet the skin surface hologram;
displaying the virtual lap arms, the laparoscopic instrument trajectories, and the virtual port markers through the augmented reality display system; and
storing a port placement plan including positions of the virtual lap arms, the virtual port markers, and the laparoscopic instrument trajectories.

14. The method of claim 13, further comprising automatically generating initial positions for the multiple virtual lap arms based on predetermined port configurations.

15. The method of claim 14, wherein automatically generating initial positions includes automatically locating an umbilicus on the skin surface hologram and positioning a first virtual lap arm through the umbilicus.

16. The method of claim 13, further comprising:
enabling interactive adjustment of the virtual lap arms by allowing a user to move the distal end of each virtual lap arm while the proximal end remains anchored at the anatomical target; and
automatically calculating and updating virtual port locations by determining intersections with the skin surface hologram in real-time as the virtual lap arms are adjusted.

17. The method of claim 13, further comprising:
performing collision detection between the laparoscopic instrument trajectories and the three-dimensional representation of the anatomical structure to identify intersections with critical anatomical structures; and
displaying a warning when a virtual lap arm is positioned within a threshold distance of a critical anatomical structure.

18. The method of claim 13, further comprising selectively displaying specific anatomical structures with adjustable transparency in response to voice commands.

19. The method of claim 13, further comprising:
retrieving the stored port placement plan during a laparoscopic procedure; and
displaying the virtual port markers and laparoscopic instrument trajectories overlaid on the patient at planned insertion locations to provide guidance for laparoscopic instrument insertion.

20. A method of augmented reality guided visualization for laparoscopic procedures, comprising:
tracking a position and an orientation of each one of a laparoscopic instrument and an ultrasound probe using a tracking system during a laparoscopic procedure;
receiving tracking data from the tracking system at a computer system;
receiving ultrasound imaging data from an ultrasound probe at the computer system;
determining an imaging plane position and orientation of the ultrasound probe based on the tracking data;
generating an ultrasound flashlight projection displaying the ultrasound imaging data positioned in three-dimensional space corresponding to the imaging plane;
determining an instrument axis for the laparoscopic instrument based on the tracking data and calibration data associated with the laparoscopic instrument;
generating a holographic light ray projection extending along the instrument axes;
displaying, through an augmented reality display system, the ultrasound flashlight projection and the holographic light ray projections; and
continuously updating the ultrasound flashlight projection and the holographic light ray projection as the laparoscopic instruments and the ultrasound probe move during the procedure.
